(19) United States
(12) Patent Application Publication
(10) Pub. No.: US 2026/0042843 A1
(43) Pub. Date: Feb. 12, 2026

(54) METHODS FOR TREATING ENDOMETRIAL AND OVARIAN HYPERPROLIFERATIVE DISORDERS

(71) Applicant: TEMPLE THERAPEUTICS BV, Geleen (NL)

(72) Inventor: Ghassan SAED, Detroit, MI (US)

(73) Assignee: TEMPLE THERAPEUTICS BV, Geleen (NL)

(21) Appl. No.: 19/099,979

(22) PCT Filed: Aug. 2, 2023
(86) PCT No.: PCT/IB2023/057843

§ 371 Date: Jan. 30, 2025

Related U.S. Application Data
(60) Provisional application No. 63/394,445, filed on Aug. 2, 2022.

(51) Int. Cl. C07K 16/28; A61K 31/337; A61K 31/4375; A61K 31/454; A61K 31/502; A61K 31/506; A61K 31/517; A61K 33/243; A61K 38/12; A61K 39/00; A61K 39/395; A61P 35/00; C07K 16/40; C12N 15/113; G01N 33/574; G01N 33/68

(52) U.S. Cl. CPC C07K 16/2842; A61K 31/337; A61K 31/4375; A61K 31/454; A61K 31/502; A61K 31/506; A61K 31/517; A61K 33/243; A61K 38/12; A61K 39/39558; A61P 35/00; C07K 16/2839; C07K 16/40; C12N 15/1137; C12Y 111/02002; G01N 33/57449; G01N 33/689; A61K 2039/507; C07K 2317/73; C12N 2310/14; G01N 2333/7055; G01N 2800/364

(57) ABSTRACT

The disclosure provides for methods and compositions that target the interaction of αV/β1 integrin and monomeric MPO in ovarian cancer and other hyperproliferative disorders. Accordingly, aspects of the disclosure relate to a method for treating endometrial or ovarian hyperproliferative disorders in a subject, the method comprising administration of an inhibitor of αV/β1 integrin, an MPO inhibitor, and/or the combination of an αV/β1 integrin and an MPO inhibitor to the subject. In some aspects, the hyperproliferative disorder comprises ovarian cancer or epithelial ovarian cancer. In some aspects, the hyperproliferative disorder comprises endometriosis.

### CROSS REFERENCE TO RELATED APPLICATIONS

This application claims the benefit of priority of U.S. Provisional Application No. 63/394,445, filed Aug. 2, 2022. The contents of the referenced application are incorporated into the present application by reference.

### BACKGROUND

### I. Field of the Invention

This invention relates to the field of medicine and molecular biology

### II. Background

It is estimated that about 21,750 women will receive a new diagnosis of ovarian cancer and about 13,940 women will die from ovarian cancer in the year of 2020. Ovarian cancer ranks fifth in cancer deaths among women, accounting for more deaths than any other cancer of the female reproductive system. A woman's risk of getting ovarian cancer during her lifetime is about 1 in 78. Her lifetime chance of dying from ovarian cancer is about 1 in 108. These statistics don't count low malignant potential ovarian tumors. Because there are no screening tests for ovarian cancer, and symptoms are often subtle, most ovarian cancer cases are diagnosed at a more advanced stage.

For stages II, III, or IV ovarian cancer, surgery is typically performed prior to chemotherapy in order to remove the tumor, both ovaries, and affected organs and lymph nodes throughout the body. After surgery, the majority of patients will start a personalized chemotherapy plan, including one or more chemotherapy drugs. Sometimes, once a diagnosis of ovarian cancer has been made by a biopsy, a few cycles of chemotherapy are given first in order to make surgery more successful and less complicated. Chemotherapy is then completed after surgery. There are some targeted or biologic therapies available for ovarian cancer. Despite the advances in ovarian cancer therapies, the 5-year relative survival rate is less than 50%. Therefore, there is a need for additional ovarian cancer therapeutics.

### SUMMARY

The disclosure provides for methods and compositions that target or measure the interaction of αV/β1 integrin and monomeric MPO in ovarian cancer and other hyperproliferative disorders.

Accordingly, the disclosure relates to a method for treating endometrial or ovarian hyperproliferative disorders in a subject, the method comprising administering an inhibitor of αV/β1 integrin, an MPO inhibitor, and/or the combination of an αV/β1 integrin and an MPO inhibitor to the subject. The hyperproliferative disorder may comprise ovarian cancer or epithelial ovarian cancer. The hyperproliferative disorder may comprise endometriosis. Also described is a method for evaluating a subject comprising detecting αV/β1 integrin in a biological sample from the subject. Also disclosed is a method for treating a subject with an endometrial or ovarian hyperproliferative disorder, the method comprising administering an treatment for the endometrial or ovarian hyperproliferative disorder to a subject that has had the level of αV/β1 integrin evaluated in a biological sample from the subject. Other methods involving administering an inhibitor of αV/β1 integrin, an MPO inhibitor, and/or the combination of an αV/β1 integrin and an MPO inhibitor to the subject include, but are not limited to, reducing or relieving symptoms of endometriosis or an ovarian hyperproliferative disorder in a subject, treating infertility in a subject, treating chemosensitive cancer, treating chemoresistant cancer, and/or treating docetaxel and/or cisplatin resistant ovarian cancer.

Also provided is a method of diagnosing a subject with an endometrial or ovarian hyperproliferative disorder comprising: a) evaluating or measuring αV/β1 integrin in a biological sample from the subject; b) comparing the measured level to control level or control samples; and c) diagnosing the subject with an endometrial or ovarian hyperproliferative disorder based on the measured level of αV/β1 integrin. Described is a method for monitoring a subject being treated for an endometrial or ovarian hyperproliferative disorder with a therapeutic agent, the method comprising a) evaluating αV/β1 integrin in a biological sample from the subject; b) comparing the measured level to control level or control samples; and c) determining the efficacy of the therapeutic agent based on the measured level of αV/β1 integrin. Also described is a method for making a complex comprising contacting a biological sample with an antibody that binds to αV/β1 integrin. Methods include methods for making a complex comprising contacting a biological sample with an antibody that binds to aV integrin, β1 integrin, and/or mMPO. Also provided is a kit comprising one or more anti-aV integrin, anti-β1 integrin, and/or αV/β1 integrin antibodies or antigen binding fragments thereof.

Methods of the disclosure include prognosing, diagnosing, or monitoring subject having an endometrial or ovarian hyperproliferative disorder, suspected of having endometrial or ovarian hyperproliferative disorder, and/or having symptoms of endometrial or ovarian hyperproliferative disorder. The subject may be diagnosed as having a stage I, II, III, or IV cancer based on the evaluated level of αV/β1 integrin.

The term hyperproliferative disorder refers to a disorder that in characterized by an abnormal growth or hyperproliferation of tissue. The hyperproliferative disorder may include or exclude hyperplasia, dysplasia, and pre-cancerous lesions. The hyperproliferation may be further characterized by a tumor. The hyperproliferation may be further defined as benign, meaning that it is non-cancerous. The hyperproliferation may be further defined as malignant, which refers to a cancerous growth.

The inhibitor may comprise or exclude an anti-αV/β1 integrin, anti-aV integrin, anti-β1 integrin antibody, an anti-MPO antibody or combinations thereof. The inhibitor may be one that inhibits the interaction of αV/β1 integrin and monomeric MPO (mMPO). The inhibitor may comprise an oligonucleotide inhibitor. The oligonucleotide inhibitor may comprise an isolated oligonucleotide that hybridizes with a nucleic acid molecule encoding the MPO, αV integrin, and/or β1 integrin gene. The oligonucleotide inhibitor may be further defined as a siRNA, a double stranded RNA, a short hairpin RNA, or an antisense oligonucleotide. The inhibitor may comprise or exclude one or more of GLPG0187, Bexotegrast (PLN-74809), PLN-1474, Abituzumab, Intetumumab, Volociximab, or Cilengitide. The inhibitor may comprise GLPG0187. The inhibitor may comprise Bexotegrast. The inhibitor may comprise PLN-1474. The inhibitor may comprise Abituzumab. The inhibitor may comprise Intetumumab. The inhibitor may comprise Volociximab. The inhibitor may comprise Cilengitide.

The subject may be one that has or has been determined to have ovarian or endometrial cells that are positive for expression of monomeric MPO and/or αV/β1 integrin. The subject may be further defined as a human subject. The subject may be a female subject. The subject may be one that has female organs. Female organs include the external genitals, or the vulva, and the internal reproductive organs, which include the ovaries and uterus. The subject may be one that is on hormone therapy or exclude one that is on hormone therapy. The hormone therapy may comprise or exclude contraception or hormone replacement therapy. The female subject may include or exclude an adolescent, perimenopausal, or menopausal female. The subject may include or exclude one that has received one or more prior therapies to treat the hyperproliferative disorder. The subject may include or exclude one that has been determined to be a non-responder or resistant to the one or more prior therapies. The one or more therapies may comprise an immunotherapy. The immunotherapy may include or exclude a checkpoint inhibitor therapy. The immunotherapy may comprise Pembrolizumab. The prior therapy may include or exclude an additional agent described herein. The one or more prior therapies may include or exclude a targeted antibody. The targeted antibody in the methods and compositions of the disclosure may be Bevacizumab. The prior therapy may comprise or further comprise or may exclude a chemotherapy. The chemotherapy may comprise or exclude a platinum-based chemotherapy, a taxane-based chemotherapy, or a combination thereof. The chemotherapy may comprise or further comprise or exclude carboplatin, paclitaxel, or a combination thereof. The prior therapy may comprise cisplatin. The prior therapy may comprise docetaxel. The method may comprise or further comprise or may exclude administration of an additional agent. The additional agent may comprise or further comprise a platinum-based chemotherapy, a taxane-based chemotherapy, or a combination thereof. Platinum-based chemotherapies useful in the methods and compositions of the disclosure include or exclude cisplatin, oxaliplatin, nedaplatin, triplatin tetranitrate, phenanthriplatin, picoplatin, satraplatin, and carboplatin. Taxane-based chemotherapies useful in the methods and compositions of the disclosure include or exclude paclitaxel, docetaxel, and cabazitaxel. The additional agent may comprise cisplatin. The additional agent may comprise docetaxel. The subject may be sensitive to or may have been determined to be sensitive to platinum-based chemotherapy and/or taxane based chemotherapy. The platinum-based chemotherapy may comprise carboplatin and/or the taxane-based chemotherapy comprises paclitaxel. The platinum-based chemotherapy may comprise cisplatin and/or the taxane-based chemotherapy may comprise docetaxel. The additional agent may comprise or exclude a PARP inhibitor, a targeted antibody, or combinations thereof. The PARP inhibitor may comprise or exclude olaparib or niraparib. The subject may be one that is or has been determined to be resistant to platinum-based chemotherapy and/or taxane based chemotherapy. The subject may be administered an inhibitor in combination with platinum-based chemotherapy when the subject is or has been determined to be sensitive to platinum-based chemotherapy. The subject may be one that is administered an inhibitor in combination with a PARP inhibitor when the subject has been determined to be resistant to platinum-based chemotherapy or in platinum resistant refractory subjects. The subject may be one that is administered an inhibitor in combination with a PARP inhibitor and bevacizumab when the subject has been determined to be resistant to platinum-based chemotherapy or in platinum resistant refractory subjects.

The cancer may include or exclude a stage I, II, III, or IV cancer, such as a stage I, II, III, or IV cancer. The cancer may be further defined as metastatic. The cancer may be further defined as recurrent. The cancer may be further defined as non-metastatic. The cancer may be further defined as non-recurrent.

Compositions of the disclosure may comprise additional agents described herein. The composition may comprise or further comprise or exclude a chemotherapeutic agent, a PARP inhibitor, a targeted antibody, or combinations thereof. Immunotherapies, chemotherapies, targeted antibodies, and additional agents may be any molecule described herein, such as a platinum-based chemotherapeutic agent, a taxane based chemotherapeutic agent, a PARP inhibitor, olaparib, niraparib, bevacizumab, cisplatin, oxaliplatin, nedaplatin, triplatin tetranitrate, phenanthriplatin, picoplatin, satraplatin, carboplatin, paclitaxel, docetaxel, and cabazitaxel.

The subject or patient of the methods of the disclosure may be one who has been diagnosed with cancer. The subject may also be one that is suspected of having a cancer but has not been diagnosed. The subject may be one that has been diagnosed with stage I cancer, diagnosed with stage II cancer, diagnosed with stage III cancer, or diagnosed with stage IV cancer. The subject may be one that has been diagnosed with metastatic cancer or has not been diagnosed with metastatic cancer. The subject may be one that has been diagnosed with recurrent cancer or has not been diagnosed with recurrent cancer. The cancer may include breast, ovarian, or head and neck cancer. The cancer may be one that is listed herein.

The biological sample may comprise or exclude serum, plasma, or tissue samples. The biological sample may be a blood sample or a fraction thereof. The biological sample may be a biological sample described herein. The biological sample may be a serum sample. The biological sample may be a plasma sample.

The methods may include or exclude evaluating iron levels in a biological sample from the subject. The evaluation of the iron level may be combined with the evaluation of αV/β1 integrin to provide a diagnosis or prognosis for the subject. The method may further comprise or may exclude detecting iron levels in the biological sample from the subject. Detecting iron levels in the biological sample from the subject may comprise one or more of a serum iron test, free iron test, a total iron-binding capacity (TIBC) test, or a ferritin test. In methods of the disclosure, the subject may be one or exclude one that has been evaluated for iron levels or such subjects may be excluded. The subject may be one or exclude one that has been evaluated for iron levels by having one or more of a serum iron test, a total iron-binding capacity (TIBC) test, or a ferritin test in a biological sample from the subject. The subject may be one or exclude one that has been determined to have abnormal iron levels. The subject may be one or exclude one that has been determined to have a lower than normal elevated TIBC. The subject may be one or exclude one that has been determined to have elevated ferritin. The subject may be one or exclude one that has been determined to have elevated serum iron. The subject may be one or exclude one that has been determined to have normal iron levels. The normal range for TIBC is 250 to 450 mcg/dL. The subject may be one that has been determined to have a TIBC of less than 250 meg/dL. The normal range for ferritin is: 20 to 250 ng/ml for adult males, 10 to 120 ng/ml for adult females, 18 to 39 years, and 12 to 263 ng/ml for females 40 years and older. The ferritin may be determined to be greater than 120 ng/ml, 250 ng/ml, or 263 ng/mL. The subject may be one or exclude one that is determined to have abnormal transferrin saturation. The subject may be one t or exclude one that is determined to have elevated transferrin saturation. Normal ranges for transferrin saturation is 20-50%. The subject may be one that has determined to have or the biological sample has greater than 50% transferrin saturation. The subject may be one or exclude one that has been determined to have, has, or the biological sample from the subject has elevated serum iron levels. The normal range for serum iron is 60 to 170 micrograms per deciliter (mcg/dL), or 10.74 to 30.43 micromoles per liter (micromol/L). The subject may be one or exclude one that has been determined to have, or the biological sample may have a serum iron level of greater than 170 mcg/dL.

The TIBC in the biological sample may be, the TIBC in the subject may have been determined to be, and/or the TIBC in a biological sample from the subject may be one or exclude one that was determined to be great than, less than, at least, or at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, or 550 mcg/dL, or any derivable range therein. The ferritin in the biological sample may be, the ferritin in the subject may be one or exclude one that is determined to be, and/or the ferritin in a biological sample from the subject may have determined to be great than, less than, at least, or at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or 350 ng/ml, or any range derivable therein. The transferrin saturation in the biological sample may be, the transferrin saturation in the subject may be one or exclude one that has been determined to be, or the transferrin saturation in a biological sample from the subject may have been determined to be great than, less than, at least, or at most 5, 10, 15, 20, 25, 30, 35, 40, 45, 50, 55, 60, 65, 70, 75, 80, 85, 90, or 95% (or any derivable range therein). The transferrin saturation in the biological sample may be, the transferrin saturation in the subject may be one or exclude one that has been determined to be, or the transferrin saturation in a biological sample from the subject may have been determined to be great than, less than, at least, or at most 30, 40, 50, 60, 70, 80, 90, 100, 110, 120, 130, 140, 150, 160, 170, 180, 190, 200, 210, 220, 230, 240, 250, 260, or 270 micrograms per deciliter, or any derivable range therein. The free iron in the biological sample may be, the free iron in the subject may be one or exclude one that has been determined to be, or the free iron in a biological sample from the subject may have been determined to be great than, less than, at least, or at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, or 550 mcg/dL, or any derivable range therein. The higher than normal level of free iron in the biological sample from the subject may be greater than 100, greater than 110, greater than 120, greater than 130, greater than 140, greater than 150 mcg/dL, or any derivable range therein.

Detecting αV/β1 integrin may comprise immunological detection of αV/β1 integrin. Detecting or evaluating αV/β1 integrin may comprise or exclude an enzyme-linked immunosorbent assay (ELISA) assay. An Elisa assay uses a solid-phase type of enzyme immunoassay (EIA) to detect the presence of a ligand (commonly a protein) in a liquid sample using antibodies directed against the protein to be measured. Antigens from the biological sample or fraction thereof may be attached to a surface. Then, an antibody may be applied over the surface so it can bind to its target from the biological sample. This antibody may be linked to a detection molecule, such as an enzyme, and then any unbound antibodies may be removed. In the final step, the detection molecule may be detected qualitatively or quantitatively. In the aspect in which the detection molecule is an enzyme, the enzyme's substrate may be added, leading to a reaction that produces a detectable signal, most commonly a color change that can be quantitatively or qualitatively measured.

The ELISA may be further characterized as a sandwich ELISA or exclude a sandwich ELISA. An antibody may be immobilized on a solid support, such as a microtiter plate or a polystyrene microtiter plate. The biological sample or fraction may be added to the solid support to allow binding between the antibody and antigen in the biological sample or fraction. Unbound molecules may be washed away from the solid support. After the antigen is immobilized, the detection antibody can be added, forming a complex with the antigen. The detection antibody can be covalently linked to a detection molecule, such as an enzyme or can itself be detected by a secondary antibody that is linked to a detection molecule, such as an enzyme. Between each step, the plate is typically washed with a mild detergent solution to remove any proteins or antibodies that are non-specifically bound. After the final wash step, the plate is developed by qualitatively or quantitatively detecting the detection molecule. In the case of enzymatic detection, the final step may comprise adding an enzymatic substrate to produce a visible signal that can qualitatively or quantitatively detected.

The ELISA may be performed using other forms of ligand binding assays instead of strictly immunoassays. An ELISA may be one that comprises any ligating reagent that can be immobilized on the solid phase along with a detection reagent that will bind specifically and use a detectable molecule to generate a signal that can be properly quantified. In between the washes, only the ligand and its specific binding counterparts remain specifically bound or “immunosorbed” by antigen-antibody interactions to the solid phase, while the nonspecific or unbound components are washed away.

“Detectable labels, molecules, or moieties” or “detection molecules, labels, or moieties” are used interchangeably and refer to compounds and/or elements that can be detected due to their specific functional properties, and/or chemical characteristics, the use of which allows the antibody to be detected, and/or further quantified if desired. Examples of detectable labels include or exclude radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels include or exclude horseradish peroxidase (HRP), fluorescein, FITC, rhodamine, dansyl, umbelliferone, dimethyl acridinium ester (DMAE), Texas red, luminol, NADPH and α-or-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include or exclude urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

The anti-MPO, anti-αV integrin, anti-bl integrin, and/or anti-aVb1 integrin antibody or binding molecule may be linked to a solid support. Suitable solid phase supports or carriers include any support capable of binding an antigen or an antibody. Exemplary supports or carriers include or exclude glass, polystyrene, polypropylene, polyethylene, dextran, nylon, amylases, natural and modified celluloses, polyacrylamides, gabbros, and magnetite. The methods of the disclosure may comprise or further comprise or may exclude washing the solid support to remove unbound molecules. The methods may comprise or further comprise or may exclude contacting the biological sample or the fraction with a capture antibody or antigen-binding molecule. The capture antibody or antigen-binding molecule may comprise or exclude a second antibody or antigen-binding fragment. Exemplary antigen-binding fragments include, for example, a single chain variable fragment (scFv), F(ab′) 2, Fab′, Fab, Fv, or rIgG. The capture antibody may be linked to a detectable label. The method may comprise or further comprise or may exclude quantitatively or qualitatively evaluating the detectable label.

The subject or patient may be a human subject or a human patient. The subject or patient may be a non-human animal. The non-human animal may be a bat, monkey, camel, rat, mouse, rabbit, goat, chicken, bird, cat, dog. The subject may further be defined as a high risk subject. The subject may include or exclude one that has one or more symptoms of endometriosis and/or ovarian cancer. Symptoms of ovarian cancer may include or exclude abdominal bloating or swelling, quickly feeling full when eating, weight loss, discomfort in the pelvis area, changes in bowel habits, such as constipation, and a frequent need to urinate. The subject may also include or exclude one that has been diagnosed with an endometrial or ovarian hyperproliferative disorder. The subject may include or exclude one that has been treated for an endometrial or ovarian hyperproliferative disorder. The subject may include or exclude one that will be treated for an endometrial or ovarian hyperproliferative disorder. The subject may include or exclude one that is currently undergoing treatment for an endometrial or ovarian hyperproliferative disorder. The subject may be further defined as a human subject. The subject may be a female. The subject may include or exclude a subject on hormone therapy. The hormone therapy may comprise or exclude contraception or hormone replacement therapy. The female subject may include or exclude an adolescent, perimenopausal, or menopausal female. The subject may include or exclude one that has one or more symptoms of endometrial or ovarian hyperproliferative disorders. The subject may be a human subject. The subject may be less than 18 years. The subject may be more than 18 years old. The subject may be, be at least, or be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 years in age, or any range derivable therein.

The method may comprise or further comprise or exclude quantitating the level of αV/β1 integrin in the biological sample. The level of αV/β1 integrin in the biological sample or fraction may be a level that has been quantitated. The level of αV/β1 integrin may include or exclude normalized. The level of αV/β1 integrin may be compared to a control. The level of αV/β1 integrin may be determined to be greater than the control. The level of αV/β1 integrin may be determined to be less than the control. The subject may be one that has or has been determined to have a level of αV/β1 integrin in the biological sample that is greater than the level of a αV/β1 integrin in a control sample. The subject may include or exclude one that has or has been determined to have a level of αV/β1 integrin in the biological sample that is less than the level of a αV/β1 integrin in a control sample. The subject may include or exclude one that has or has been determined to have a level of αV/β1 integrin in the biological sample that is not significantly different than the level of a αV/β1 integrin in a control sample. For example, the level of αV/β1 integrin may be determined to be, to be at least, or to be at most 0.5, 1, 1.5, 2, 2.5, 3, 3.5, 4, or 5 standard deviations different than or within a control value. The level of αV/β1 integrin may be determined to be, to be at least, or to be at most 1, 2, 5, 10, 15, 20, 25, 30, 35, 40, 45, 50, 55, 60, 65, 70, 75, 80, 85, 90, 95, 98, 99, or 100% (or any derivable range therein) above or below a control level of αV/β1 integrin. The control may comprise the level of αV/β1 integrin that is representative of the level of αV/β1 integrin in a biological sample from a subject with endometriosis. The control may comprise the level of αV/β1 integrin that is representative of the level of αV/β1 integrin in a biological sample from a subject with ovarian cancer. The control may comprise the level of αV/β1 integrin that is representative of the level of αV/β1 integrin in a biological sample from a subject without an endometrial or ovarian hyperproliferative disorders. The method may further comprise diagnosing the subject. The subject may be diagnosed with ovarian cancer based on the determined level of αV/β1 integrin. The subject may include or exclude one that is diagnosed with or the cancer comprises stage I, II, III, or IV ovarian cancer based on the determined level of αV/β1 integrin. The methods of the disclosure may also comprise or further comprise treating the subject for ovarian cancer. The subject may be diagnosed with endometriosis based on the determined level of αV/β1 integrin. The methods of the disclosure may comprise or further comprise treating the subject for endometriosis.

The therapeutic agent may be determined to be effective when αV/β1 integrin: i) is not detected in the biological sample from the subject; ii) is not significantly different than a control, wherein the control comprises the level of αV/β1 integrin that is representative of the level of αV/β1 integrin in a biological sample from a subject without an endometrial or ovarian hyperproliferative disorders; iii) is less than a control, wherein the control comprises the level of αV/β1 integrin that is representative of the level of αV/β1 integrin in a biological sample from a subject with an endometrial or ovarian hyperproliferative disorder; or iv) is decreased compared to the level of αV/β1 integrin before treatment of the subject with the therapeutic agent. The therapeutic agent may be determined to be ineffective when αV/β1 integrin: i) detected in the biological sample from the subject; ii) is increased compared to a control, wherein the control comprises the level of αV/β1 integrin that is representative of the level of αV/β1 integrin in a biological sample from a subject without an endometrial or ovarian hyperproliferative disorders; iii) is not significantly different or more than a control, wherein the control comprises the level of αV/β1 integrin that is representative of the level of αV/β1 integrin in a biological sample from a subject with an endometrial or ovarian hyperproliferative disorder; or iv) is not significantly different or increased compared to the level of αV/β1 integrin before treatment of the subject with the therapeutic agent.

The method may further comprise evaluating the level of αV/β1 integrin in a biological sample from the subject obtained prior to treatment. The method may further comprise evaluating the level of αV/β1 integrin in a biological sample from the subject obtained after one or more treatments. For example, the method may comprise or further comprise evaluating αV/β1 integrin levels after one dose, after two doses, after three doses, after four doses, after five doses, and/or after six doses of a particular treatment. Treatment efficacy may be determined based on the evaluated levels of αV/β1 integrin.

The level of αV/β1 integrin may be determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml. (or any derivable range therein) in the biological sample from the subject. The subject may be one that is diagnosed with Stage I cancer when the level of αV/β1 integrin is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. The subject may be diagnosed with Stage II cancer when the level of αV/β1 integrin is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. The subject may be diagnosed with Stage III cancer when the level of αV/β1 integrin is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml. (or any derivable range therein) in the biological sample from the subject. The subject may be diagnosed with Stage IV cancer when the level of αV/β1 integrin is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. Late stage cancer may comprise Stage IV and/or Stage III. Early stage cancer may comprise Stage I and/or Stage II.

The control level may comprise the level of αV/β1 integrin that is representative of the level of αV/β1 integrin in a subject with endometriosis. The control may comprise the level of αV/β1 integrin that is representative of the level of αV/β1 integrin in a subject with ovarian cancer. The control may comprise the level of αV/β1 integrin that is representative of the level of αV/β1 integrin in a subject without an endometrial or ovarian hyperproliferative disorders. The hyperproliferative disorder may comprise endometriosis. The hyperproliferative disorder may comprise ovarian cancer.

The treatment may be one known in the art for endometriosis or ovarian cancer or one described herein. The treatment may comprise or exclude hormone therapy, or surgery. Other treatments useful in the methods of the disclosure include or exclude surgery, radiation, chemotherapy, hormone therapy, immunotherapy, or targeted therapy.

The subject may be diagnosed as not having an endometrial or ovarian hyperproliferative disorder when αV/β1 integrin: i) is not detected in the biological sample from the subject; ii) is not significantly different than a control, wherein the control comprises the level of αV/β1 integrin that is representative of the level of αV/β1 integrin in a biological sample from a subject without an endometrial or ovarian hyperproliferative disorders; or iii) is less than a control, wherein the control comprises the level of αV/β1 integrin that is representative of the level of αV/β1 integrin in a biological sample from a subject with an endometrial or ovarian hyperproliferative disorder. The subject may be diagnosed as having endometriosis when αV/β1 integrin: i) is greater than a control, wherein the control comprises the level of αV/β1 integrin that is representative of the level of αV/β1 integrin in a biological sample from a subject without an endometrial or ovarian hyperproliferative disorders; ii) is not significantly different than the control; wherein the control comprises the level of αV/β1 integrin that is representative of the level of αV/β1 integrin in a biological sample from a subject with endometriosis; or iii) is less than a control, wherein the control comprises the level of αV/β1 integrin that is representative of the level of αV/β1 integrin in a biological sample from a subject with ovarian cancer. The subject may be diagnosed as having ovarian cancer when αV/β1 integrin: i) is greater than a control, wherein the control comprises the level of αV/β1 integrin that is representative of the level of αV/β1 integrin in a biological sample from a subject without an endometrial or ovarian hyperproliferative disorders or from a subject with endometriosis; ii) is not significantly different than a control, wherein the control comprises the level of αV/β1 integrin that is representative of the level of αV/β1 integrin in a biological sample from a subject with ovarian cancer. Evaluating αV/β1 integrin may comprise qualitatively detecting the level of αV/β1 integrin in the biological sample. The subject may be diagnosed as not having an endometrial or ovarian hyperproliferative disorder when αV/β1 integrin in not detected in the biological sample from the subject. The subject may be diagnosed as having an endometrial or ovarian hyperproliferative disorder when αV/β1 integrin is detected in the biological sample from the subject.

Kits of the disclosure may comprise or further comprise one or more negative or positive control samples. The kit may comprise an ELISA for detecting αV/β1 integrin. The antibody or binding fragment may be operatively linked to a solid support. The kit may comprise at least two antibodies, at least two antibody binding fragments, or one antibody and one antibody binding fragment. The one or more antibodies or antibody binding fragments may be linked to a detectable label.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments and aspects described herein in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments and aspects discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments and aspects are discussed throughout this application. Any embodiment or aspect discussed with respect to one aspect of the disclosure applies to other aspects of the disclosure as well and vice versa.

Other objects, features and advantages of the present invention will become apparent from the following detailed description. It should be understood, however, that the detailed description and the specific examples, while indicating specific embodiments or aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B. The expression of integrin αV and integrin β1 in sensitive and chemoresistant EOC cell lines. Integrin αV or integrin β1 protein levels were determined in sensitive EOC cell lines (SKOV-3, TOV-21G, and A2780) and their docetaxel or cisplatin resistant counterparts utilizing immunoprecipitation (IP)/Western blot (A). Integrin αV or integrin β1 protein was immunoprecipitated from EOC cell lysates with anti-integrin αV or anti-integrin β1 antibody and fractionated with SDS-PAGE (B). Membranes were probed with anti-integrin αV or anti-integrin β1 antibody. IP/Western blot results were scanned and analyzed by NIH image J 3.0. All experiments were performed in triplicate. * Indicates p<0.05 as compared to their sensitive counterpart. Each set of three bars in FIG. 1B corresponds to data from (left to right) A2780, TOV-21G, and MDAH-2774 cells, respectively.

FIG. 2A-D. Integrins αV and B1 are localized to the cell surface in sensitive and chemoresistant EOC cells. Localization of integrin αV and integrin β1 to the cell surface was confirmed in EOC cells and macrophages with flow cytometry. There was insignificant expression of integrin αV, while integrin β1 was significantly expressed in macrophages.

FIG. 3A-B. Integrins αV and B1 exist as a heterodimer that binds MPO. A proximity ligand assay was used to demonstrate the dimerization of αV and B1 in SKOV-3 and TOV112D EOC cells (A), as well as the heterodimer's binding to MPO (B). Interaction of integrins αV and B1 was determined utilizing the PLA, in which the individual proteins were immunolabeled first with two primary antibodies and then with different species-specific secondary antibodies conjugated to complementary oligonucleotides. If the two antibody molecules are in close proximity, complementary DNA strands can be ligated, amplified and visualized with a fluorescent probe as individual points.

FIG. 4A-B. The integrin αV and integrin β1 antibodies are cytotoxic to sensitive and chemoresistant EOC cell lines, and this effect is synergistic with standard chemotherapy. Cytotoxicity (A) was determined in human macrophages (n=2) (first box in each group of four boxes), as well as in sensitive (n=5) EOC cell lines (second box in each group of four boxes) and their docetaxel (third box in each group of four boxes) or cisplatin resistant (fourth box in each group of four boxes) (n=5) counterparts by the MTT Cell Proliferation Assay. Significant cytotoxicity was observed in both sensitive and chemoresistant EOC cells as compared to their isotype controls and macrophages. Synergy (B) of the integrin αV and β1 antibodies (0.5 or 1.5 μg/ml) was determined following combination of each antibody with increasing doses of cisplatin (0.1, 0.5, 1.0 μM) and analyzed by the automated calculation of combination index (CI) values will be conducted by the CompuSyn software where CI<1 is synergistic, CI=1 is additive, and CI>1 is antagonistic.

FIG. 5A-B. Chemoresistant ovarian cancer cells have lower levels of apoptosis as compared to their sensitive counterparts. Caspase-3 activity (A) was measured in cell lysates from sensitive EOC cell lines (SKOV-3, A2780, and MDAH-2774) as compared to their docetaxel or cisplatin resistant counterparts. S-nitrosylation of caspase-3 (B) in EOC cells, SKOV-3 before (1) and after (2) silencing MPO gene expression with specific siRNA, and MDAH-2774 before (3) and after (4) silencing iNOS gene expression with specific siRNA probes. All experiments were performed in triplicate. * Indicates p<0.05 as compared to their sensitive counterpart. Each group of three bars in FIG. 5A represents data, from left to right, for MDAH-2774, SKOV-3, and A2780 cell lines. Each group of two bars in FIG. 5B represents data, from left to right, for MDAH-2774 and SKOV-3 cell lines.

FIG. 6. Proposed mechanism of survival in EOC cells. The proposed model illustrates that the binding of MPO to αV/β1 activates MPO in EOC cells. Activated MPO utilizes nitric oxide (NO), produced by iNOS, as a one-electron substrate to generate nitrosonium cation (NO+), a labile nitrosylating species that increases S-nitrosylation of caspase-3 and inhibits apoptosis and thus increases survival. NO=nitrous oxide; NO+=nitrosium cation; iNOS=inducible nitric oxide synthase, MPO=myeloperoxidase

### DETAILED DESCRIPTION OF THE INVENTION

The inventors have identified a cell membrane receptor, αV/β1 integrin, that is uniquely expressed by both chemosensetive and chemoreistant EOC cells with significantly higher expression in chemoresistant EOC cells. Furthermore, they have demonstrated that monoclonal antibodies against αV/β1 integrin induced cytotoxicity in EOC cells, but not in normal cells, that is also synergistic with conventional chemotherapies. Cytotoxicity of αV/β1 antibodies is due to conformational changes in αV/β1 integrin which prevents monomeric MPO binding to αV/β1 integrin inhibiting the activation of MPO leading to increased apoptosis. Since normal epithelial cells and macrophages lack monomeric MPO and αV/β1 integrin system, targeting this unique MPO-dependent survival mechanism will selectively eliminate EOC cells and will be the target for developing specific ovarian cancer therapies.

### I. Inhibitors

### A. Inhibitory Oligonucleotides

The disclosure provides for inhibitory oligonucleotides that inhibit the gene expression of an integrin and/or MPO. Examples of an inhibitory oligonucleotides include but are not limited to siRNA (small interfering RNA), short hairpin RNA (shRNA), double-stranded RNA, an antisense oligonucleotide, a ribozyme and a oligonucleotide encoding thereof. An inhibitory oligonucleotide may inhibit the transcription of a gene or prevent the translation of a gene transcript in a cell. An inhibitory oligonucleotide acid may be from 16 to 1000 nucleotides long or from 18 to 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory oligonucleotide may be capable of decreasing the expression of the protein by at least 10%, 20%, 30%, or 40%, more particularly by at least 50%, 60%, or 70%, and most particularly by at least 75%, 80%, 90%, 95%, 99%, or 100% more or any range or value in between the foregoing.

Also described are synthetic oligonucleotides that are alpha-V, beta-1, or MPO inhibitors. An inhibitor may be between 17 to 25 nucleotides in length and comprises a 5′ to 3′ sequence that is at least 90% complementary to the 5′ to 3′ sequence of a mature mRNA. An inhibitor molecule may be 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

The inhibitory oligonucleotide may be an analog and my include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure His modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros-a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. The oligonucleotide may be further defined as having one or more locked nucleotides, ethylene bridged nucleotides, peptide nucleic acids, or a 5′ (E)-vinyl-phosphonate (VP) modification. The oligonucleotides may have one or more phosphorothioated DNA or RNA bases.

### B. Antibodies

An antibody or a fragment thereof may be one that binds to at least a portion of αV/β1 integrin, αV integrin, β1 integrin, and/or MPO protein and modulates it's activity, such as its binding activity, enzymatic activity, or binding specificity.

Also described are antibodies comprising a heavy or light chain, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE. IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4:302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (2). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the—COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgAQ1 and IgA2.

Antibodies can be whole immunoglobulins of any isotype or classification, chimeric antibodies, or hybrid antibodies with specificity to two or more antigens. They may also be fragments (e.g., F(ab′) 2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:
The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. Bispecific antibodies can be biparatopic, wherein a bispecific antibody may specifically recognize a different epitope from the same antigen. Bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

The antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

Multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the extrenal surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

Affinity matured antibodies may be enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thic et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

Portions of the heavy and/or light chain may be identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

Minimizing the antibody polypeptide sequence from the non-human species may optimize chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. Sec, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhocyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### 1. Functional Antibody Fragments and Antigen-Binding Fragments

Also provided are antibody fragments, such as antibody fragments that bind to and modulate activity. The term functional antibody fragment includes antigen-binding fragments of an antibody that retain the ability to specifically bind to an antigen. These fragments are constituted of various arrangements of the variable region heavy chain (VH) and/or light chain (VL); and include constant region heavy chain 1 (CH1) and light chain (CL). They may lack the Fc region constituted of heavy chain 2 (CH2) and 3 (CH3) domains. Antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′) 2 fragment means a bivalent antigen-binding fragment of a monoclonal antibody comprising two Fab′ fragments linked by a disulfide bridge at the hinge region. An F(ab′) 2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv) 2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv) 2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### Fragment Crystallizable Region, Fc

A fragment crystallizable region (Fc region) contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

Antigen-binding peptide scaffolds, such as complementarity-determining regions (CDRs), are used to generate protein-binding molecules. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### II. Hyperproliferative Disorder Treatments

Methods and compositions may be provided for treating hyperproliferative disorders with particular applications of biomarker expression or activity levels. Based on a profile of biomarker expression or activity levels, different treatments may be prescribed or recommended for different patients. The hyperproliferative disorder may comprise ovarian cancer or a cancer originating in the ovarian epithelium, germ cells, or stroma, fallopian epithelium, ovaries, cervix, fallopian tube, or uterus. The hyperproliferative disorder may be primary peritoneal carcinoma, fallopian tube cancer, teratoma, dysgerminoma, or a yolk sac tumor. The cancer may comprise a cancer stage, TNM, and/or is further characterized as having features described below.

### A. Cancer Staging

The most common staging system is the TNM (for tumors/nodes/metastases) system, from the American Joint Committee on Cancer (AJCC). The TNM system assigns a number based on three categories. “T” denotes the degree of invasion of the intestinal wall, “N” the degree of lymphatic node involvement, and “M” the degree of metastasis. The broader stage of a cancer is usually quoted as a number I, II, III, IV derived from the TNM value grouped by prognosis; a higher number indicates a more advanced cancer and likely a worse outcome. Details of this system are in the tables below:

| AJCC/ | Stage | |
|---|---|---|
| FIGO | Grouping | Stage Description* |
| I | T1 | The cancer is only in the ovary (or ovaries) or fallopian tube(s) (T1). |
| | N0 | It has not spread to nearby lymph nodes (N0) or to distant sites (M0). |
| | M0 | |
| IA | T1a | The cancer is in one ovary, and the tumor is confined to the inside of the ovary; |
| | N0 | or the cancer is in one fallopian tube, and is only inside the fallopian tube. |
| | M0 | There is no cancer on the outer surfaces of the ovary or fallopian tube. No |
| | | cancer cells are found in the fluid (ascites) or washings from the abdomen and |
| | | pelvis (T1a). |
| IB | T1b | The cancer is in both ovaries or fallopian tubes but not on their outer surfaces. |
| | N0 | No cancer cells are found in the fluid (ascites) or washings from the abdomen |
| | M0 | and pelvis (T1b). It has not spread to nearby lymph nodes (N0) or to distant |
| IC | T1c | The cancer is in one or both ovaries or fallopian tubes and any of the following |
| | N0 | are present: |
| | M0 | The tissue (capsule) surrounding the tumor broke during surgery, |
| | | which could allow cancer cells to leak into the abdomen and pelvis |
| | | (called surgical spill). This is stage IC1. |
| | | Cancer is on the outer surface of at least one of the ovaries or fallopian |
| | | tubes or the capsule (tissue surrounding the tumor) has ruptured (burst) |
| | | before surgery (which could allow cancer cells to spill into the |
| | | abdomen and pelvis). This is stage IC2. |
| II | T2 | The cancer is in one or both ovaries or fallopian tubes and has spread to other |
| | N0 | organs (such as the uterus, bladder, the sigmoid colon, or the rectum) within |
| | M0 | the pelvis or there is primary peritoneal cancer (T2). It has not spread to nearby |
| IIA | T2a | The cancer has spread to or has invaded (grown into) the uterus or the fallopian |
| | N0 | tubes, or the ovaries. (T2a). It has not spread to nearby lymph nodes (N0) or to |
| | M0 | distant sites (M0). |
| IIB | T2b | The cancer is on the outer surface of or has grown into other nearby pelvic |
| | N0 | organs such as the bladder, the sigmoid colon, or the rectum (T2b). It has not |
| | M0 | spread to nearby lymph nodes (N0) or to distant sites (M0). |
| IIIA1 | T1 or T2 | The cancer is in one or both ovaries or fallopian tubes, or there is primary |
| | N1 | peritoneal cancer (T1) and it may have spread or grown into nearby organs in |
| | M0 | the pelvis (T2). It has spread to the retroperitoneal (pelvic and/or para-aortic) |
| IIIA2 | T3a | The cancer is in one or both ovaries or fallopian tubes, or there is primary |
| | N0 or N1 | peritoneal cancer and it has spread or grown into organs outside the pelvis. |
| | M0 | During surgery, no cancer is visible in the abdomen (outside of the pelvis) to |
| | | the naked eye, but tiny deposits of cancer are found in the lining of the |
| | | abdomen when it is examined in the lab (T3a). |
| | | [symbol] |
| IIIB | T3b | There is cancer in one or both ovaries or fallopian tubes, or there is primary |
| | N0 or N1 | peritoneal cancer and it has spread or grown into organs outside the pelvis. The |
| | M0 | deposits of cancer are large enough for the surgeon to see, but are no bigger |
| | | than 2 cm (about ¾ inch) across. (T3b). |
| | | It may or may not have spread to the retroperitoneal lymph nodes (N0 or N1). |
| IIIC | T3c | The cancer is in one or both ovaries or fallopian tubes, or there is primary |
| | N0 or N1 | peritoneal cancer and it has spread or grown into organs outside the pelvis. The |
| | M0 | deposits of cancer are larger than 2 cm (about ¾ inch) across and may be on |
| | | the outside (the capsule) of the liver or spleen (T3c). |
| | | It may or may not have spread to the retroperitoneal lymph nodes (N0 or N1). |
| IVA | Any T | Cancer cells are found in the fluid around the lungs (called a malignant pleural |
| | Any N | effusion) with no other areas of cancer spread such as the liver, spleen, |
| | M1a | intestine, or lymph nodes outside the abdomen (M1a). |
| IVB | Any T | The cancer has spread to the inside of the spleen or liver, to lymph nodes other |
| | Any N | than the retroperitoneal lymph nodes, and/or to other organs or tissues outside |
| | M1b | the peritoneal cavity such as the lungs and bones (M1b). |

[symbol]

indicates data missing or illegible when filed

The “cancer” referred to in the methods described herein may include or exclude any of the above stages or TNM categories. The “cancer” referred to in the methods described herein may include or exclude any of the above stages or TNM categories. For example the cancer may be or may exclude Stage 0, I, IA, IB, IC, II, IIA, IIB, IIIA1, IIIA2, IIIB, IIIC, IVA, or IVB cancer. The patient may be one that has and/or has been determined to have Stage 0, I, IA, IB, IC, II, IIA, IIB, IIIA1, IIIA2, IIIB, IIIC, IVA, or IVB cancer. Furthermore, the cancer may be stage NO and/or MO; T1, NO, and/or MO; T1, N1, and/or M0; T2, NO, and/or M0; T1, N2, and/or MO; T2, N1, and/or MO; T3, NO, and/or MO; T1, N3, and/or MO; T2, N2, and/or MO; T3, N1, and/or MO; T4a, NO, and/or MO; T2, N3, and/or M0; T3, N2, and/or MO; T4a, N1, and/or MO; T3, N3, and/or MO; T4a, N2, and/or MO; T4b and/or NO; N1 and/or MO; T4a, N3, and/or MO; T4b and/or N2; N3 and/or MO; Any T, any N, and/or M1.

### B. Treatments

Methods of the disclosure relate to treating subjects and patients with a cancer therapy. The cancer therapy may be one described below and may be given with respect to a patient having been determined to have a certain biomarker profile. For example, the therapy described below may be given to a patient with a poor prognosis, unfavorable prognosis, or to a patient determined to be high risk. The therapy described below may be given to a patient with a favorable prognosis, or to a patient determined to be low risk. Also contemplated are combinations of the therapies described below.

The cancer treatment may be surgery, radiation, chemotherapies, hormone therapies, or targeted therapies. The radiation may be further characterized as external beam radiation therapy or brachytherapy. The chemotherapy may be a platinum compound and/or a taxane. Platinum compounds include cisplatin and carboplatin. Taxanes include paclitaxel and docetaxel. The chemotherapy may comprise a combination of a chemotherapeutic platinum compound and a taxane. Further chemotherapies include albumin bound paclitaxel, altretamine, capecitabine, cyclophosphamide, etoposide, gemcitabine, ifosfamide, irinotecan, liposomal doxorubicin, melphalan, pemetrexed, topotecan, and vinorelbine.

Common ways to give chemotherapy include an intravenous (IV) tube placed into a vein using a needle or in a pill or capsule that is swallowed (orally). A chemotherapy regimen usually comprises a specific number of cycles given over a set period of time. A patient may receive 1 drug at a time or combinations of different drugs at the same time.

Antimetabolites can be used in cancer treatment, as they interfere with DNA production and therefore cell division and the growth of tumors. Because cancer cells spend more time dividing than other cells, inhibiting cell division harms tumor cells more than other cells. Anti-metabolites masquerade as a purine (azathioprine, mercaptopurine) or a pyrimidine, chemicals that become the building-blocks of DNA. They prevent these substances becoming incorporated in to DNA during the S phase (of the cell cycle), stopping normal development and division. They also affect RNA synthesis. However, because thymidine is used in DNA but not in RNA (where uracil is used instead), inhibition of thymidine synthesis via thymidylate synthase selectively inhibits DNA synthesis over RNA synthesis. Due to their efficiency, these drugs are the most widely used cytostatics. In the ATC system, they are classified under L01B.

Thymidylate synthase inhibitors are chemical agents which inhibit the enzyme thymidylate synthase and have potential as an anticancer chemotherapy. As an anti-cancer chemotherapy target, thymidylate synthetase can be inhibited by the thymidylate synthase inhibitors such as fluorinated pyrimidine fluorouracil, or certain folate analogues, the most notable one being raltitrexed (trade name Tomudex). Additional agents include pemetrexed, nolatrexed, ZD9331, and GS7904L.

Also described are prodrugs that can be converted to thymidylate synthase inhibitors in the body, such as Capecitabine (INN), an orally administered chemotherapeutic agent used in the treatment of numerous cancers. Capecitabine is a prodrug, that is enzymatically converted to 5-fluorouracil in the body.

If cancer has entered the lymph nodes, adding the chemotherapy agents fluorouracil or capecitabine increases life expectancy. Chemotherapy agents for this condition may include capecitabine, fluorouracil, irinotecan, leucovorin, oxaliplatin and UFT. Another type of agent that is sometimes used are the epidermal growth factor receptor inhibitors.

Alternative treatments may be prescribed or recommended based on the biomarker profile. In addition to traditional chemotherapy for gastric cancer patients, cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Just as for chemotherapy, radiotherapy can be used in the neoadjuvant and adjuvant setting for some stages of cancer.

Targeted therapy may also be used in the methods described herein. Targeted therapies include angiogenesis inhibitors such as bevacizumab and/or PARP inhibitors such as, olaparib, rucaparib, and/or niraparib. Also included are NTRK targeted drugs, such as Larotrectinib and entrectinib.

Hormone therapies include luteinizing-hormone-releasing agonists, tamoxifen, and aromatase inhibitors.

Immunotherapies that are designed to boost the body's natural defenses to fight the cancer may also be used. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting.

### C. Monitoring

The biomarker-based method may be combined with one or more other cancer diagnosis or screening tests at increased frequency if the patient is determined to be at high risk for recurrence or have a poor prognosis based on the biomarker as described above.

The methods of the disclosure may further include one or more monitoring tests. The monitoring protocol may include any methods known in the art. In particular, the monitoring may include obtaining a sample and testing the sample for diagnosis. For example, the monitoring may include endoscopy, biopsy, laparoscopy, colonoscopy, blood test, genetic testing, endoscopic ultrasound, X-ray, barium enema x-ray, chest x-ray, barium swallow, a CT scan, a MRI, a PET scan, or HER2 testing. The monitoring test may comprise radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

### D. ROC Analysis

In statistics, a receiver operating characteristic (ROC), or ROC curve, is a graphical plot that illustrates the performance of a binary classifier system as its discrimination threshold is varied. ROC analysis may be applied to determine a cut-off value or threshold setting of biomarker expression. For example, patients with biological samples determined to have biomarker expression value above a certain cut-off threshold but below a higher cut-off threshold may be determined to have endometriosis. Patients with biological samples determined to have a biomarker expression level that surpasses the cut-off threshold for endometriosis may be determined to have cancer. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1-specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz CE (1978) Basic principles of ROC analysis. Seminars in Nuclear Medicine 8:283-298; Youden WJ (1950) An index for rating diagnostic tests. Cancer 3:32-35; Zweig MH, Campbell G (1993) Receiver-operating characteristic (ROC) plots: a fundamental evaluation tool in clinical medicine. Clinical Chemistry 39:561-577; and Greiner M, Pfeiffer D, Smith RD (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### III. Additional Agents

### A. Immunostimulators

The method may further comprise administration of an additional agent. The additional agent may be an immunostimulator. The term “immunostimulator” as used herein refers to a compound that can stimulate an immune response in a subject, and may include an adjuvant. An immunostimulator may be an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalenc+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

The additional agent may comprise an agonist for pattern recognition receptors (PRR), including, but not limited to Toll-Like Receptors (TLRs), specifically TLRs 2, 3, 4, 5, 7, 8, 9 and/or combinations thereof. Additional agents may comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. The additional agents also may comprise immunostimulatory RNA molecules, such as but not limited to dsRNA, poly I:C or poly I:poly C12U (available as Ampligen.RTM., both poly I:C and poly I:polyC12U being known as TLR3 stimulants), and/or those disclosed in F. Heil et al., “Species-Specific Recognition of Single-Stranded RNA via Toll-like Receptor 7 and 8” Science 303 (5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. An additional agent may be a TLR-4 agonist, such as bacterial lipopolysaccharide (LPS), VSV-G, and/or HMGB-1. Additional agents may comprise TLR-5 agonists, such as flagellin, or portions or derivatives thereof, including but not limited to those disclosed in U.S. Pat. Nos. 6,130,082, 6,585,980, and 7,192,725.

Additional agents may be proinflammatory stimuli released from necrotic cells (e.g., urate crystals). Additional agents may be activated components of the complement cascade (e.g., CD21, CD35, etc.). Additional agents may be activated components of immune complexes. Additional agents also include complement receptor agonists, such as a molecule that binds to CD21 or CD35. The complement receptor agonist may induce endogenous complement opsonization of the synthetic nanocarrier. Immunostimulators may be cytokines, which are small proteins or biological factors (in the range of 5 kD-20 kD) that are released by cells and have specific effects on cell-cell interaction, communication and behavior of other cells. The cytokine receptor agonist may be a small molecule, antibody, fusion protein, or aptamer.

### B. Immunotherapies

The additional therapy may comprise a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### 1. Inhibition of Co-Stimulatory Molecules

The immunotherapy may comprise an inhibitor of a co-stimulatory molecule. The inhibitor may comprise an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7. TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta).

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons αre produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 6. Checkpoint Inhibitors and Combination Treatment

The additional therapy may comprise immune checkpoint inhibitors. Certain aspects are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. PD-1, PDL1, and PDL2 may be further defined as human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may be PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

The immune checkpoint inhibitor may be a PDL1 inhibitor such as Durvalumab, also known as MEDI4736, atezolizumab, also known as MPDL3280A, avelumab, also known as MSB00010118C, MDX-1105, BMS-936559, or combinations thereof. The immune checkpoint inhibitor may be a PDL2 inhibitor such as rHIgM12B7.

The inhibitor may comprise the heavy and light chain CDRs or VRs of nivolumab, pembrolizumab, or pidilizumab. Accordingly, the inhibitor may comprise the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may be one that blocks the CTLA-4 and B7-1 interaction. The inhibitor may be one that blocks the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

The inhibitor may comprise the heavy and light chain CDRs or VRs of tremelimumab or ipilimumab. Accordingly, the inhibitor may comprise the CDR1, CDR2, and CDR3 domains of the VH region of tremelimumab or ipilimumab, and the CDR1, CDR2 and CDR3 domains of the VL region of tremelimumab or ipilimumab. The antibody may be one that competes for binding with and/or binds to the same epitope on PD-1, B7-1, or B7-2 as the above-mentioned antibodies. The antibody may be one that has at least about 70, 75, 80, 85, 90, 95, 97, or 99% (or any derivable range therein) variable region amino acid sequence identity with the above-mentioned antibodies.

### C. Oncolytic Virus

The additional therapy may comprise an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### D. Polysaccharides

The additional therapy may comprise polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### E. Neoantigens

The additional therapy may comprise neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### F. Chemotherapies

The additional therapy may comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated. The amount of cisplatin delivered to the cell and/or subject, when used in combination with the inhibitors described herein, may be less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. Appropriate intravenous doses for an adult may include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### G. Radiotherapy

The additional therapy or prior therapy may comprise radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

The amount of ionizing radiation may be greater than 20 Gy and is administered in one dose. The amount of ionizing radiation may be 18 Gy and may be administered in three doses. The amount of ionizing radiation may be at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). The ionizing radiation may be administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of IR may be presented as a total dose of IR, which is then administered in fractionated doses. For example, the total dose is 50 Gy administered in 10 fractionated doses of 5 Gy each. The total dose may be 50-90 Gy, administered in 20-60 fractionated doses of 2-3 Gy each. The total dose of IR may be at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40.41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses may be administered (or any derivable range therein). At least, at most, or exactly 1, 2, 3, 4, 5, 6, 7, 8, 9, 10, 11, or 12 (or any derivable range therein) fractionated doses may be administered per day. At least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses may be administered per week.

### H. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the inhibitors of the disclosure, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### I. Other Agents

It is contemplated that other agents may be used in combination with certain aspects of the disclosure to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. Cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present disclosure to improve the treatment efficacy.

### IV. Analysis of Gene Expression Levels

A gene shall be understood to be specifically expressed in a certain cell type if the expression level of said gene in said cell type is at least 2-fold, 5-fold, 10-fold, 100-fold, 1000-fold, or 10000-fold higher than in a reference cell type, or in a mixture of reference cell types. Reference cell types include non-cancerous tissue cells or a heterogeneous population of cancers.

Comparison of multiple marker genes with a threshold level can be performed as follows: 1. The individual marker genes are compared to their respective threshold levels. 2. The number of marker genes, the expression level of which is above their respective threshold level, is determined. 3. If a marker genes is expressed above its respective threshold level, then the expression level of the marker gene is taken to be “above the threshold level”.

The determination of expression levels may be done on a gene chip, such as an Affymetrix™ gene chip. The determination of expression levels may be done by kinetic real time PCR.

The methods can relate to a system for performing such methods, the system comprising (a) apparatus or device for storing data on the biomarker level of the patient; (b) apparatus or device for determining the expression level of at least one marker gene or activity; (c) apparatus or device for comparing the expression level of the first marker gene or activity with a predetermined first threshold value; (d) apparatus or device for determining the expression level of at least one second, third, fourth, 5th, 6th or more marker gene or activity and for comparing with a corresponding predetermined threshold; and (c) computing apparatus or device programmed to provide a unfavorable or poor prognosis or favorable prognosis based on the comparisons.

The person skilled in the art readily appreciates that an unfavorable or poor prognosis can be given if the expression level of the first marker gene with the predetermined first threshold value indicates a tumor that is likely to recur or not respond well to standard therapies.

The expression patterns can also be compared by using one or more ratios between the expression levels of different cancer biomarkers. Other suitable measures or indicators can also be employed for assessing the relationship or difference between different expression patterns.

The expression levels of cancer biomarkers can be compared to reference expression levels using various methods. These reference levels can be determined using expression levels of a reference based on all cancer patients. Alternatively, it can be based on an internal reference such as a gene that is expressed in all cells. The reference may be a gene expressed in cancer cells at a higher level than any biomarker. Any comparison can be performed using the fold change or the absolute difference between the expression levels to be compared. One or more cancer biomarkers can be used in the comparison. It is contemplated that 1, 2, 3, 4, 5, 6, 7, 8, 9, 10, and/or 11 biomarkers (or any range derivable therein) may be compared to each other and/or to a reference that is internal or external. A person of ordinary skill in the art would know how to do such comparisons.

Comparisons or results from comparisons may reveal or be expressed as x-fold increase or decrease in expression relative to a standard or relative to another biomarker or relative to the same biomarker but in a different class of prognosis. Patients with a poor prognosis may have a relatively high level of expression (overexpression) or relatively low level of expression (underexpression) when compared to patients with a better or favorable prognosis, or vice versa.

Fold increases or decreases may be, be at least, or be at most 1-, 2-, 3- , 4-, 5-, 6-, 7-, 8-, 9-, 10-, 11-, 12-, 13-, 14-, 15-, 16-, 17-, 18-, 19-, 20-. 25-, 30-, 35-, 40-. 45-, 50-. 55-, 60-. 65-. 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are with normalized or relative numbers such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein. The levels can be relative to a control.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered.

Any biological sample from the patient that contains cancer cells may be used to evaluate the expression pattern of any biomarker discussed herein. A biological sample from a tumor may be used. Evaluation of the sample may involve, though it need not involve, panning (enriching) for cancer cells or isolating the cancer cells.

### A. Measurement of Gene Expression Using Nucleic Acids

Testing methods based on differentially expressed gene products are well known in the art. The differential expression patterns of cancer biomarkers may be determined by measuring the levels of RNA transcripts of these genes, or genes whose expression is modulated by the these genes, in the patient's cancer cells. Suitable methods for this purpose include, but are not limited to, RT-PCR, Northern Blot, in situ hybridization, Southern Blot, slot-blotting, nuclease protection assay and oligonucleotide arrays.

RNA isolated from cancer cells can be amplified to cDNA or cRNA before detection and/or quantitation. The isolated RNA can be either total RNA or mRNA. The RNA amplification can be specific or non-specific. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. Detection may involve fluorescence resonance energy transfer (FRET) or some other kind of quantum dots.

Amplification primers or hybridization probes for a cancer biomarker can be prepared from the gene sequence or obtained through commercial sources, such as Affymatrix. The gene sequence may be identical or complementary to at least 8 contiguous nucleotides of the coding sequence.

Sequences suitable for making probes/primers for the detection of their corresponding cancer biomarkers include those that are identical or complementary to all or part of the cancer biomarker genes described herein. These sequences are all nucleic acid sequences of cancer biomarkers.

The use of a probe or primer of between 13 and 100 nucleotides, particularly between 17 and 100 nucleotides in length, or up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Each probe/primer may comprise at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. Because each of the biomarkers may have more than one human sequence, it is contemplated that probes and primers may be designed for use with each of these sequences. For example, inosine is a nucleotide frequently used in probes or primers to hybridize to more than one sequence. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

The probes/primers for a gene may be selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W. T, and X determine the sensitivity and speed of the alignment. These parameters can be adjusted for different purposes, as appreciated by one of ordinary skill in the art.

Quantitative RT-PCR (such as TaqMan, ABI) may be used for detecting and comparing the levels of RNA transcripts in cancer samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable cDNAs may be normalized to some independent standard, which may be based on either internally existing RNA species or externally introduced RNA species. The abundance of a particular mRNA species may also be determined relative to the average abundance of all mRNA species in the sample.

The PCR amplification may utilize one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in clinical samples is that they are of variable quantity and/or quality. This problem can be overcome if the RT-PCR is performed as a relative quantitative RT-PCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

The relative quantitative RT-PCR may use an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

Nucleic acid arrays can also be used to detect and compare the differential expression patterns of cancer biomarkers in cancer cells. The probes suitable for detecting the corresponding cancer biomarkers can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. The density may be 50, 100, 200, 300, 400, 500 or more probes/cm².

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Also provided is the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length. The oligonucleotide probes may be 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448A1, all of which are specifically incorporated by reference.

Nuclease protection assays may be used to quantify RNAs derived from the cancer samples. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

### B. Measurement of Gene Expression Using Proteins and Polypeptides

The differential expression patterns of cancer biomarkers can be determined by measuring the levels of polypeptides encoded by these genes in cancer cells. Methods suitable for this purpose include, but are not limited to, immunoassays such as ELISA, RIA, FACS, dot blot, Western Blot, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded by the cancer biomarker genes described herein.

One example of a method suitable for detecting the levels of target proteins in peripheral blood samples is ELISA. In an exemplifying ELISA, antibodies capable of binding to the target proteins encoded by one or more cancer biomarker genes are immobilized onto a selected surface exhibiting protein affinity, such as wells in a polystyrene or polyvinylchloride microtiter plate. Then, cancer cell samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the peripheral blood samples can be lysed using various methods known in the art. Proper extraction procedures can be used to separate the target proteins from potentially interfering substances.

The cancer cell samples containing the target proteins may be immobilized onto the well surface in an ELIZA assay, and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 49° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. The label may be an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

Another suitable method is RIA (radioimmunoassay). An example of RIA is based on the competition between radiolabeled-polypeptides and unlabeled polypeptides for binding to a limited quantity of antibodies. Suitable radiolabels include, but are not limited to, 1125. A fixed concentration of 1125-labeled polypeptide may be incubated with a series of dilution of an antibody specific to the polypeptide. When the unlabeled polypeptide is added to the system, the amount of the 1125-polypeptide that binds to the antibody is decreased. A standard curve can therefore be constructed to represent the amount of antibody-bound 1125-polypeptide as a function of the concentration of the unlabeled polypeptide. From this standard curve, the concentration of the polypeptide in unknown samples can be determined. Various protocols for conducting RIA to measure the levels of polypeptides in cancer cell samples are well known in the art.

Suitable antibodies include, but are not limited to, polyclonal antibodies, monoclonal antibodies, chimeric antibodies, humanized antibodies, single chain antibodies, Fab fragments, and fragments produced by a Fab expression library.

Antibodies can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. Such technology can be used to quantitate a number of proteins in a sample, such as a cancer biomarker proteins.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

The levels of polypeptides in samples can be determined by detecting the biological activities associated with the polypeptides. If a biological function/activity of a polypeptide is known, suitable in vitro bioassays can be designed to evaluate the biological function/activity, thereby determining the amount of the polypeptide in the sample.

### V. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

Antibodies, or antibody fragments or derivatives, can be used in methods such as Western blots, ELISA, or immunofluorescence techniques to detect biomarker expression. Either the antibodies or proteins may be immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. Antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 1251, 3H, and 1311. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase,.beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

A detection label may be indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). The antibody may not be labeled, and the presence thereof can be detected using a labeled antibody, which binds to the antibody.

### VI. Sample Preparation

Methods may involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. The sample may be obtained from a biopsy from ovarian or endometrial tissue by any of the biopsy methods previously mentioned. The sample may be obtained from any of the tissues provided herein that include but are not limited to non-cancerous or cancerous tissue and non-cancerous or cancerous tissue from the ovarian epithelium, fallopian epithelium, ovaries, cervix, fallopian tube, or uterus. Alternatively, the sample may be obtained from any other source including but not limited to blood, serum, plasma, sweat, hair follicle, buccal tissue, tears, menses, feces, or saliva. Any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, or semen.

The sample may be obtained by methods known in the art. The samples may be obtained by biopsy. The sample may be obtained by swabbing, endoscopy, scraping, phlebotomy, or any other methods known in the art. The sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

The biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. A molecular profiling business may consult on which assays or tests are most appropriately indicated. The patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. Multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

The molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

A medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. Molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

The subject may be referred to a specialist such as an oncologist, surgeon, or endocrinologist. The specialist may likewise obtain a biological sample for testing or refer the individual to a testing center or laboratory for submission of the biological sample. The medical professional may refer the subject to a testing center or laboratory for submission of the biological sample. The subject may provide the sample. A molecular profiling business may obtain the sample.

### VII. Methods of Treatment

The compositions, peptides, and nucleic acids encoding the peptides of the disclosure may be used for the treatment of certain cancers. The cancer may be a αV/β1 integrin+cancer. The subject may be one that has been determined to have a αV/β1 integrin+cancer. The cancer/cancer cells have an increased expression of αV/β1 integrin compared to non-cancerous tissues of the same type. The subject may be one that has been determined to have increased expression of αV/β1 integrin in cancer cells as compared to the expression of αV/β1 integrin in non-cancerous cells.

### VIII. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). The first and second cancer treatments may be administered in a separate composition. The first and second cancer treatments may be in the same composition.

Described herein are compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. The cancer therapy may be administered intravenously, intramuscularly, subcutaneously, topically, orally, transdermally, intraperitoneally, intraorbitally, by implantation, by inhalation, intrathecally, intraventricularly, or intranasally. The antibiotic may be administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

The quantity to be administered, both according to number of treatments and unit dose, depends on the treatment effect desired. An effective dose is understood to refer to an amount necessary to achieve a particular effect. It is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

The effective dose of the pharmaceutical composition may be one which can provide a blood level of about 1 μM to 150 μM. The effective dose may be one that provides a blood level of about 4 μM to 100 μM; or about 1 μM to 100 μM; or about 1 μM to 50 μM; or about 1 μM to 40 M; or about 1 μM to 30 μM; or about 1 μM to 20 μM; or about 1 μM to 10 μM; or about 10 μM to 150 M; or about 10 μM to 100 M; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). The dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 UM or any range derivable therein. The therapeutic agent that is administered to a subject may be metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IX Kits

The disclosure provides for kits containing compositions of the invention or compositions to implement methods of the invention. Kits can be used to evaluate one or more biomarkers. A kit may contain, contain at least or contain at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, antibodies, or inhibitors, or any value or range and combination derivable therein. Also provided are kits for evaluating biomarker activity or level in a cell.

Kits may comprise components, which may be individually packaged or placed in a container, such as a tube, bottle, vial, syringe, or other suitable container means.

Individual components may also be provided in a kit in concentrated amounts. A component may be provided individually in the same concentration as it would be in a solution with other components. Concentrations of components may be provided as 1×, 2×, 5×, 10×, or 20× or more.

Kits for using probes, antibodies, synthetic nucleic acids, non-synthetic nucleic acids, and/or inhibitors of the disclosure for prognostic or diagnostic applications are included as part of the disclosure. Specifically contemplated are any such molecules corresponding to any biomarker identified herein, which includes antibodies that bind to such biomarkers as well as nucleic acid primers/primer sets and probes that are identical to or complementary to all or part of a biomarker, which may include noncoding sequences of the biomarker, as well as coding sequences of the biomarker.

Negative and/or positive control nucleic acids, antibodies, probes, and inhibitors may be included in the kit. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

### X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Binding of Intracellular Myeloperoxidase to αV/β1 Integrin Serves as a Mechanism of Survival in Epithelial Ovarian Cancer

### A. Introduction

Ovarian cancer is considered one of the most lethal gynecologic malignancies, ranking fifth in cancer deaths among women diagnosed with gynecological cancer [1]. The prognosis of epithelial ovarian cancer (EOC) remains poor, with a 5-year survival rate of 50% in advanced stages [2]. This is largely due to the lack of early warning symptoms, ineffective screening methods, and the development of chemoresistance [3]. Typically, the primary treatment of ovarian cancer includes both cytoreductive surgery and platinum/taxane combination chemotherapy. Initially, 50-80% of patients with advanced disease will achieve complete clinical response. Unfortunately, most will recur and ultimately develop chemoresistant disease [4].

Understanding the mechanisms of cell survival and chemoresistance in EOC are central to both clinical and basic molecular research in ovarian cancer chemotherapy. The objective of this work is to determine the mechanisms that support ovarian cancer cell survival. These studies will be of significant clinical importance to the detection and treatment of ovarian cancer.

### B. Materials and Methods

2.1 Cell Lines: Human EOC cell lines, MDAH-2774 (CRL-10303), SKOV-3 (HTB-77), OV90 (CRL-11732), TOV-21G (CRL-11730), TOV-112D (CRL-11731), as well as human macrophage cells (EL-1, CRL-9854) were all obtained from American Type Culture Collection (ATCC, Manassas, VA), and were all cultured with media supplemented with fetal bovine serum (FBS, Innovative Research, Novi, MI) and penicillin/streptomycin according to the manufacturers' protocols. Sensitive and cisplatin resistant TOV-112D EOC cells were a kind gift from Gen Sheng Wu at Wayne State University, Detroit, MI [26]. Chemoresistance to either docetaxel (0.3 μM) or cisplatin (1.5 μM) was achieved in ovarian cancer cell lines as previously described [23]. The A2780 human ovarian cancer cell line and its cisplatin resistant counterpart (1 μM) were obtained from Sigma Aldrich (St. Louis, MO) and cultured in RPMI-1640 (ThermoFisher Scientific) supplemented with FBS and penicillin/streptomycin as described above. Culture medium was replaced every two days. All cells were utilized between passages 3 and 5 for subsequent experiments. Mycoplasma contamination was ruled out following growth of cells on coverslips and staining with DAPI. Once confluent, cells (5×10⁶) were seeded in 150 mm dishes and were collected after 24 hours for protein extraction and isolation of RNA.

2.2 Proximity Ligand Assay: The Duolink Proximity Ligand Assay (PLA) is based on the employment of proximity probes, composed by oligonucleotide-conjugated antibodies, to recognize specific targets, and whether they bind to one another, as previously described [27]. The binding of probes in close proximity allows for their hybridization by connector oligonucleotides that can form a circular DNA strand which can then be amplified by polymerase chain reaction. Finally, the conjugation of fluorescence-labelled oligonucleotides with the amplification product allows for the localized detection of individual or interacting proteins in cells and tissues [28]. Cells were seeded on gelatin coated coverslips and fixed in 4% paraformaldehyde for 15 minutes, blocked with normal serum, and incubated overnight with mouse anti-integrin αV (Santa Cruz Biotechnology (SCBT) SC9969) and rabbit anti-MPO (Abcam, ab93665), or rabbit anti-integrin αV (Abcam, ab179475) and mouse anti-integrin β1 (SCBT, sc3744), or mouse anti-integrin β1 (SCBT, sc3744) and rabbit anti-MPO (Abcam, ab963655) at 4° C. Proximity ligation was performed according to the manufacturer's protocol using the Duolink PLA PLUS and MINUS Probes for mouse or rabbit.

2.3 Immunoprecipitation and Western Blot: Cell lysate was prepared using cell lysis buffer (Cell Signaling Technology, Danvers, MA) supplemented with a protease inhibitor (Protease Arrest; G-Biosciences, St Louis, MO). Total protein concentration of cell lysates was determined using the Pierce BCA Protein Assay kit (Thermo Scientific) according to the manufacturer's protocol. Immunoprecipitation was used for the assessment integrins αV and β1 using the Protein A/G PLUS-Agarose Immunoprecipitation Reagent (Santa Cruz Biotechnology (SCBT), Dallas, TX), according to the manufacturer's protocol. Cell lysates were precleared with mouse IgG1 (integrin αV, sc-3877, SCBT) or IgG2a isotypes (integrin β1, sc-3878, SCBT) followed by incubation with 1 μg integrin αV or integrin β1 primary antibody (sc-9969 and sc-374430 respectively, SCBT) for 1 hour at 4° C., followed by the addition of agarose beads (20 μl protein A/G PLUS-Agarose, SCBT) and incubated overnight at 4° C. Immunoprecipitates were collected by centrifugation followed by 4 washes with phosphate-buffered saline (PBS). Washed cell pellets were resuspended in 20 μl of 1× NuPAGE LDS Sample Buffer (Invitrogen) and boiled at 70° C. for 10 minutes. Samples were spun at 2500 rpm for 5 minutes at 4° C. to pellet the agarose beads.

Samples were run on a NuPAGE 4%-12% Bis-Tris Protein Gel (Invitrogen) and separated under reducing conditions as previously described [22]. Proteins were then transferred to PVDF membranes, membranes were blocked for 30 minutes with 10% bovine serum albumin (BSA) in Tris buffered saline and Tween 20 ([TBST] 20 mM Tris [pH 8.0], 150 nM NaCl, 0.05% Tween 20) at room temperature followed by incubation overnight at 4° C. with cither monoclonal antibody mouse-anti-integrin αV or mouse-anti-integrin β1 (SCBT). Membranes were washed 5 times, 5 minutes each, with TBST. Membranes were incubated with the corresponding horseradish peroxidase-conjugated secondary antibody (SCBT), diluted to 1:5000 in 2.5% BSA in TBST for 1 hour at room temperature. Membranes were washed in TBST 5 times, 5 minutes each. Membranes were developed using enhanced chemiluminescence reagent (Amersham Biosciences, General Electric Company Fairfield, CT). Image J Software (version 1.5a) was used to determine band density.

2.4 Flow cytometry: Both sensitive and chemoresistant (docetaxel or cisplatin) ovarian cancer cell lines (A2780, TOV-21G, and MDAH-2774) were collected by trypsinization and resuspended in PBS and then transferred to FACS tubes (Corning Life Sciences, Durham, NC). Whole blood cells, naive and stimulated macrophages were also analyzed. Cells were washed in 1×PBS and incubated with 20 μL of human FcR blocking reagent (Miltenyi Biotech) in 80 μL of BD FACS stain buffer (554656; BD Biosciences) for 10 min at 4° C. Next, the cells were incubated with extracellular fluorochrome-conjugated anti-human monoclonal antibodies for 30 min at 4° C. in the dark (Integrin αV Antibody (13C2) FITC (sc-53360 FITC), Integrin β1 Antibody (P5D2) PE (sc-13590 PE)). Following incubation, cells were washed with 1×PBS and then resuspended in 0.5 mL of BD FACS stain buffer, and acquired using BD LSRII Flow Cytometer (BD Bioscience) and BD FACSDiva 6.0 software (BD Bioscience) in the Microscopy, Imaging & Cytometry Resources (MICR) core from Wayne State University (http:/mier.med.wayne.edu/). Isotypes (normal mouse IgG1-PE (sc-2866), normal mouse IgG1-FITC (sc-2855) and compensation controls were also included. The analysis was performed and the figures were generated using FlowJo v10 software (FlowJo, Ashland, OR).

2.5 Determination of cytotoxicity following treatment with Integrin αV or B1 antibodies: Cytotoxicity was determined using the TACS MTT Cell Proliferation Assay (Trevigen, Gaithersburg, MD) per the manufacturer's protocol and as previously described [29,30]. Cells were seeded into Falcon tissue culture treated 96-well plates in a fixed volume of 100 μl at a density of 8,000 cells/well. Cells were treated with increasing doses (0.5 or 1.5 μg/ml, 24 hours) of either monoclonal mouse anti-integrin αV (sc-9969, SCBT) or monoclonal mouse anti-integrin β1 (sc-9970, SCBT), or the combined integrin αV+integrin β1 polyclonal rabbit anti-human (ABIN676718, Bioss Antibodies, Woburn, MA) for 24 hours. The mouse anti-IgG₁ antibody, (SCBT, sc-3877) or the rabbit anti-IgG (ABIN1881001, Bioss Antibodies) were used as isotype controls.

Determination of the synergistic effect of cisplatin and integrin αV or integrin β1 antibodies. The MTT Cell Proliferation Assay was also used to determine the combined effect of the antibodies and cisplatin in the A2780 sensitive and the A2780 cisplatin resistant EOC cell lines. Cells were treated with monoclonal mouse-anti-integrin αV antibody, mouse-anti-integrin β1, or isotype control (0, 0.5, and 1.5 μg/ml, SCBT). Cells were treated with increasing doses of cisplatin (0, 0.1, 0.5, 1.0 μM, VWR Scientific) with or without mouse-anti-integrin αV or mouse-anti-integrin β1 antibodies (0, 0.5, or 1.5 μM. SCBT) for 24 hours. For determination of synergism, dose-effect curve parameters for cisplatin and integrin αV or integrin 1 antibodies were used for the automated calculation of combination index values conducted by the CompuSyn software (ComboSyn, Paramus, NJ, USA) to determine the nature of compound interaction (antagonistic, additive or synergistic effect), as previously described [30]. Fa-combination index plots (Chou-Talalay plots) were generated by the Compusyn Software where Fa is the fraction affected (Fa=percentage of inhibition relative to vehicle control/100).

2.6 Silencing or Inhibiting MPO Expression: MPO gene expression was silenced with specific siRNA (50 nM, Ambion Silencer Select siRNA, Thermofisher Scientific, Grand Island, NY) in both sensitive and chemoresistant EOC cell lines utilizing the GenMute™ siRNA Transfection Reagent (SignaGen Laboratories, Rockville, MD) per the manufacturer's protocol. This approach has been used in our laboratory as previously described [22]. MPO activity was inhibited with melatonin (400 μM), for 24 hrs. Scrambled siRNA or dimethylformamide (DMSO) served as controls for the siRNA or melatonin, respectively.

2.7 Determination of Apoptosis: The Caspase-3 Colorimetric Activity Assay Kit (Chemicon, Temecula, CA) was used to determine levels of caspase-3 activity in sensitive and chemoresistant EOC cell lines, as previously described [22]. Equal concentrations of cell lysate were used. The assay is based on spectophotometric detection of the chromophore p-nitroaniline (pNA) after cleavage from the labeled substrate DEVD-pNA. The free pNA can be quantified using a spectrophotometer or a microtiter plate reader at 405 nm, and levels were determined based on a pNA standard.

2.8 Statistical Analysis: Data were analyzed using SPSS v23.0 (IBM, Chicago, IL) for Windows with independent sample t-tests for sensitive EOC cells as compared to their chemoresistant counterparts for Western blot and caspase-3 activity. For cytotoxicity, pair-wise differences in normally distributed variables in treatment and control groups were compared using one-way ANOVA with significant interactions further analyzed with Tukey post hoc analysis with Bonferroni correction. Box and whiskers plots indicate the 25-75th percentile by a box, whiskers show 5-95th percentiles and median value is represented as a line across each box. Unpaired t-tests were used to compare non-cancer to ovarian cancer groups, or between cisplatin and docetaxel resistant groups. The automated calculation of combination index values was conducted by the CompuSyn software (ComboSyn) for determination of synergism [21]. Statistical significance of p<0.05 was considered significant for all analyses.

### C. Results

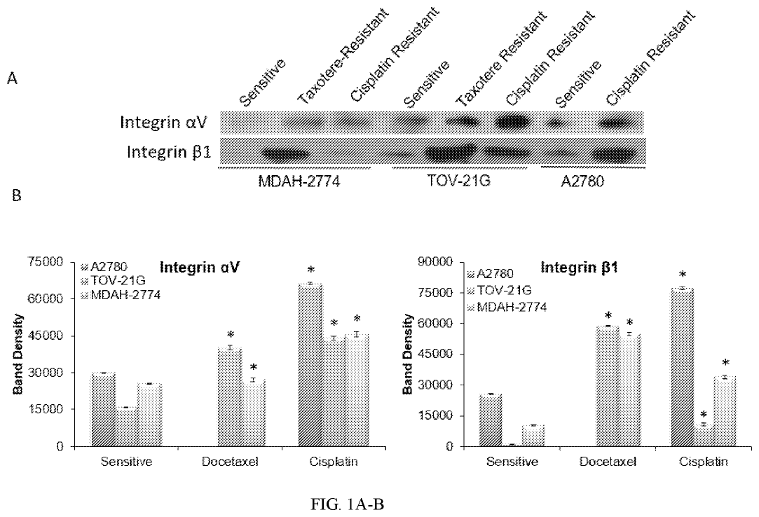

3.1 Integrin αV and β1 subunits are expressed in sensitive and chemoresistant EOC cells. The presence of integrin αV or integrin 1 subunits were determined in both sensitive and chemoresistant EOC cell lines (MDAH-2774, TOV-21G, and A2780) by Immunoprecipitation and Western blot (FIG. 1A). Cisplatin resistant A2780 EOC cells had a 55%±0.4 increase in level of integrin V compared to their sensitive counterpart. There was a 5.4%±2.2 and 43.7%±1.3 increase in integrin αV in docetaxel and cisplatin resistant MDAH-2774 EOC cells compared to their sensitive counterpart, respectively (p<0.01). There was a 60.2%±0.8 and 63.8%±0.7 increase in integrin αV in docetaxel and cisplatin resistant TOV-21G EOC cells compared to their sensitive counterpart, respectively (p<0.0001). Cisplatin resistant A2780 EOC cells had a 66.9%±0.3 increase in integrin β1 levels compared to their sensitive counterpart (p<0.0001). There was a 81.3%±0.3 and 69.8%±0.8 increase in integrin β1 levels in docetaxel and cisplatin resistant MDAH-2774 EOC cells compared to their sensitive counterparts, respectively (p<0.0001). There was a 97.8%±0.01 and 89%±0.7 increase in integrin β1 levels in docetaxel and cisplatin resistant TOV-21G EOC cells compared to their sensitive counterparts, respectively (p<0.0001, FIG. 1B).

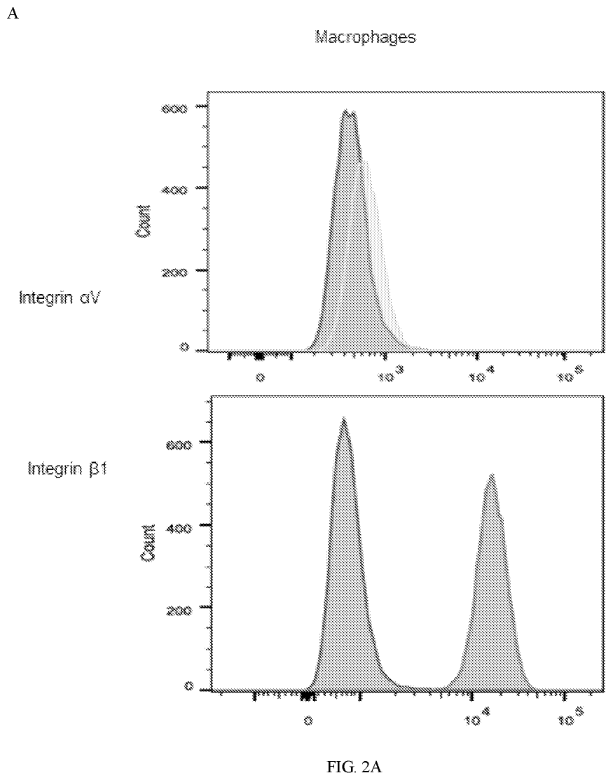

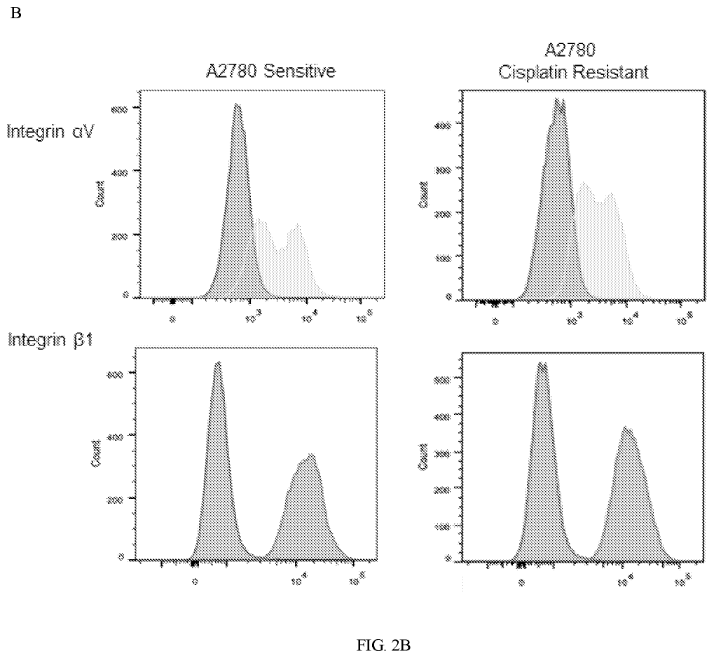

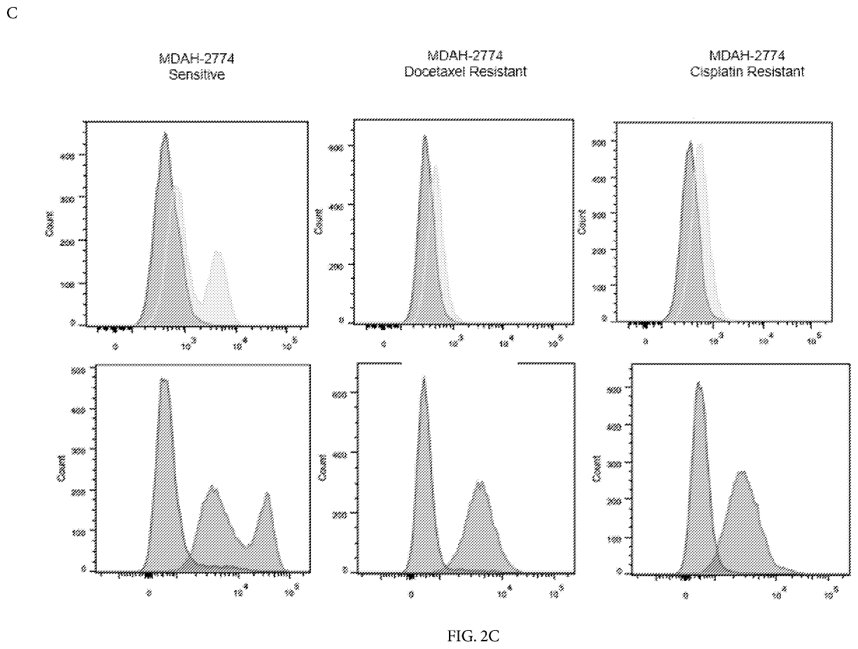

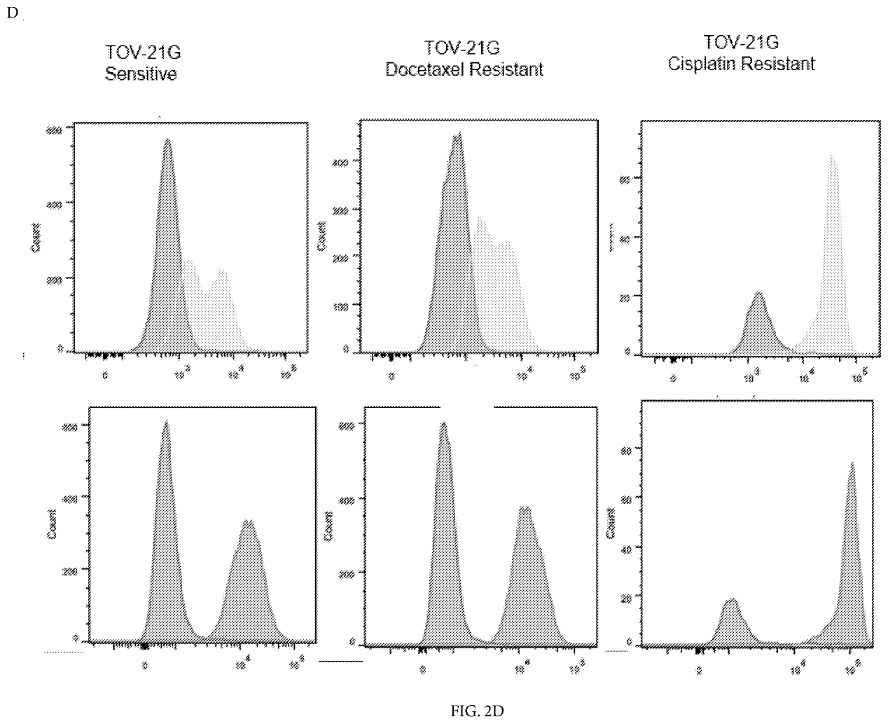

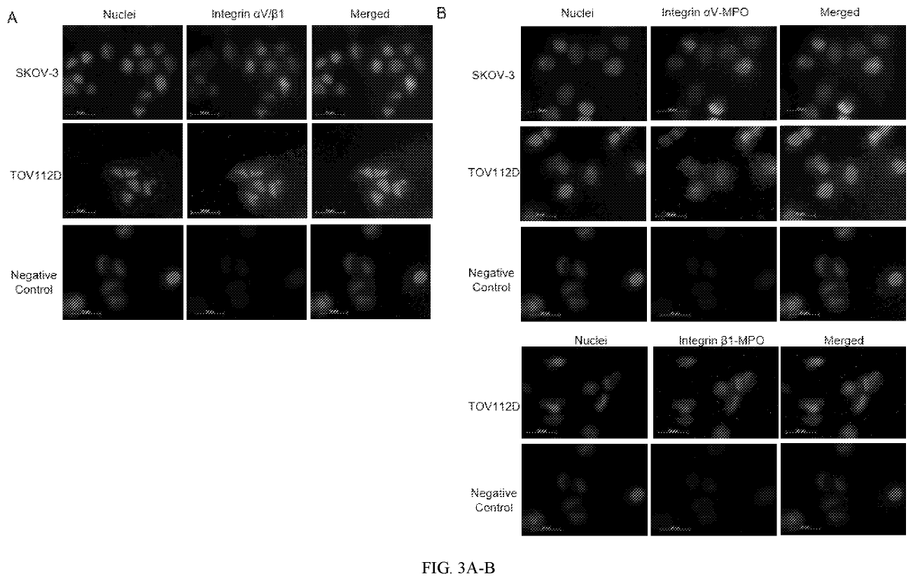

3.2 Integrin αV and β1 are localized to the cell surface in sensitive and chemoresistant EOC cells. Localization of integrin αV and integrin β1 to the cell surface was assessed in sensitive and chemoresistant EOC cell lines (MDAH-2774, TOV-21G, and A2780) as well as macrophages, by flow cytometry. Cell surface expression of both integrin αV and integrin β1 were confirmed in both sensitive and chemoresistant EOC cells; however only integrin β1 was significantly expressed in macrophages (FIG. 2). The presence of the integrins αV and β1 subunits as a heterodimer was confirmed by PLA (FIG. 3A). Integrin αV/β1 heterodimer binding to MPO was also confirmed by PLA (FIG. 3B).

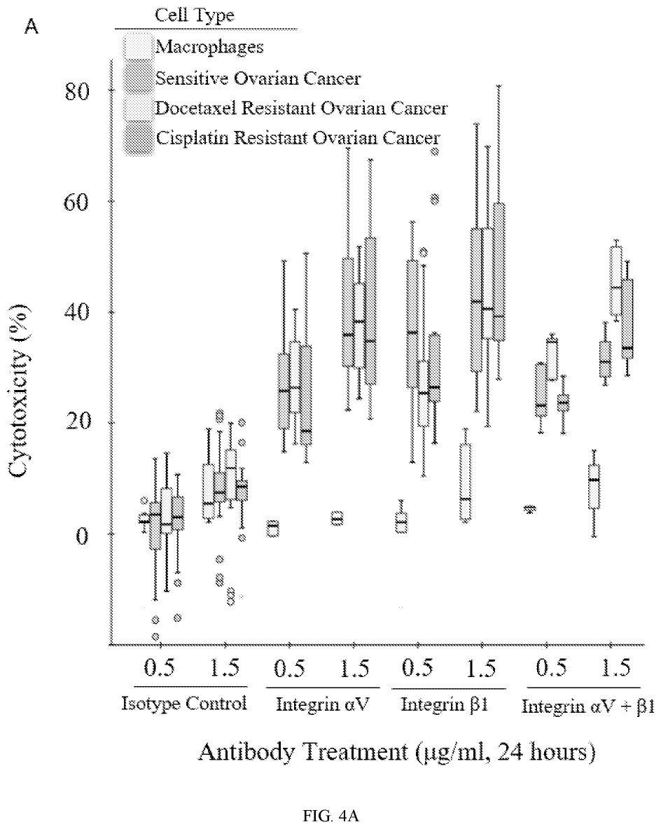

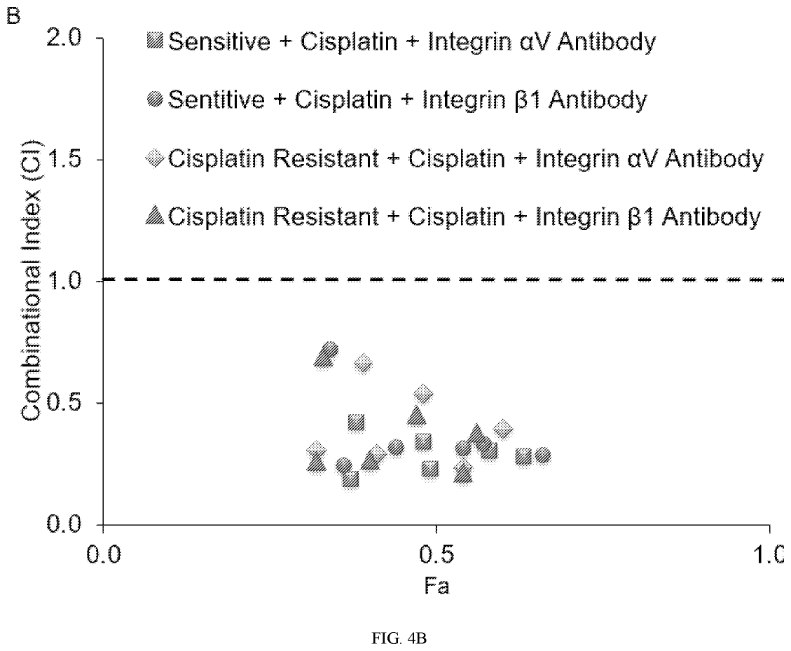

3.3 Integrin αV and integrin β1 antibodies manifested a cytotoxic effect on sensitive and chemoresistant EOC cells: Treatment with 0.5 or 1.5 μg/ml integrin αV antibody resulted in significant cytotoxicity in sensitive (27.8%±11.1 and 40.4%±14.0), docetaxel resistant (28.0%±7.7 and 37.9%±9.2), and cisplatin resistant (25.0%±12.7 and 39.8%±14.9) EOC cells as compared to isotype controls, respectively (FIG. 4A, p<0.05). Similarly, treatment with 0.5 or 1.5 μg/ml integrin β1 antibody resulted in significant cytotoxicity in sensitive (35.9%±14.2 and 43.9%±16.2), docetaxel resistant (27.8%±13.0 and 44.9%±15.2) and in cisplatin resistant (32.7%±15.3 and 46.9%±17.3) EOC cells as compared to isotype controls, respectively (p<0.05, FIG. 4A). Treatment with 0.5 or 1.5 μg/ml combined integrin αV+1 antibody resulted in significant cytotoxicity in sensitive (24.4%±5.0 and 31.8%±4.2), docetaxel resistant (32.7%±3.8 and 45.2%±6.7), and cisplatin resistant (23.4%±3.7 and 37.1%±8.0) EOC cells as compared to isotype controls, respectively (FIG. 4A, p<0.05). However, neither integrin αV, integrin β1, nor the combined αV+B1 antibody were significantly cytotoxic to macrophages (p>0.05, FIG. 4A).

Treatment of the sensitive or cisplatin resistant, but not docetaxel resistant, EOC cells with the integrin αV antibody resulted in a dose response effect (p<0.02). Moreover, there was also a dose response effect of the integrin β1 antibody in docetaxel and cisplatin resistant but not sensitive EOC cells (p<0.005). Treatment with the 1.5 μg/ml dose of integrin αV or integrin 1 antibody was significantly more cytotoxic than 0.5 μg/ml combined αV+β1 integrin antibody in both sensitive and cisplatin resistant but not docetaxel resistant EOC cell lines (p<0.02). On the other hand, treatment with 1.5 μg/ml combined αV+β1 integrin antibody was significantly more cytotoxic than either the 0.5 μg/ml integrin αV or 1.5 μg/ml integrin β1 antibody, in only docetaxel resistant EOC cells (p<0.01, FIG. 4A).

3.4 Combining integrin αV or integrin β1 antibody with cisplatin was synergistic in sensitive and chemoresistant EOC cells: The combination of the integrin αV or integrin β1 antibody (0.5 or 1.5 μg/ml) with increasing doses of cisplatin (0.1, 0.5, or 1.0 μM) resulted in a synergistic effect in both sensitive and cisplatin resistant A2780 EOC cells as compared to treatment with the antibody or cisplatin alone (Combinational index (CI)<1, p<0.05, FIG. 4B). Compusyn software generates Chou-Talalay plots where the X axis represents fractional activity (Fa) which reflects the fraction of cellular viability affected by the drug treatment relative to controls; the Y axis represents the combination index (CI) with <1, >1, and =1 indicating synergistic, antagonistic, and additive effects, respectively.

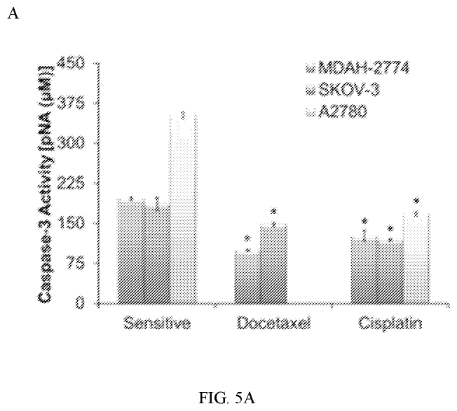

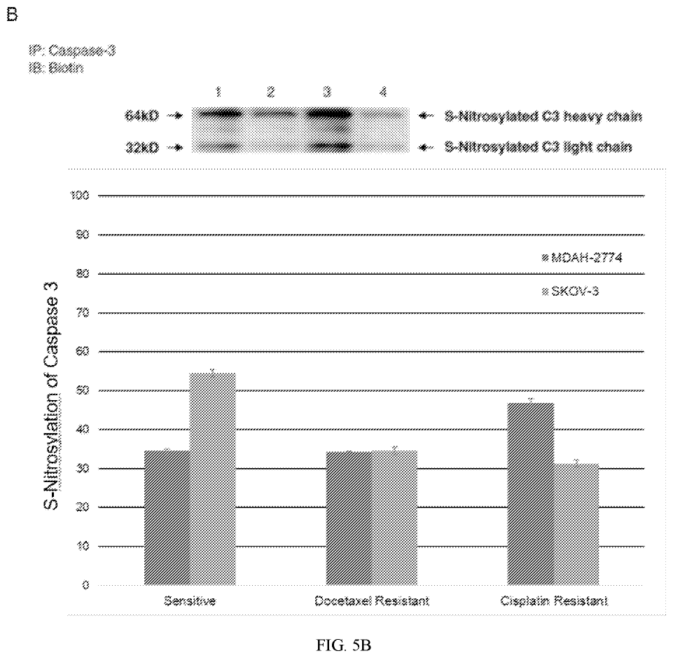

3.5 Chemoresistant EOC cells have lower levels of apoptosis: Chemoresistant EOC cells had significantly lower levels of caspase-3 activity as compared to their sensitive counterparts (FIG. 5A). There was a 49.2% and a 32.9% decrease in the levels of caspase-3 activity (from 196.3±2.8 to 99.7±1.9 and to 126.6±9.7 μM pNA) in docetaxel and cisplatin resistant MDAH-2774 EOC cells as compared to sensitive counterparts, respectively (p<0.02, FIG. 5A). Similarly, there was a 20.6% and a 36.4% decrease in the levels of caspase-3 activity (from 186.0±11.8 to 147.67±2.8 and to 118.3±1.9 μM pNA) in docetaxel and cisplatin resistant SKOV-3 EOC cells as compared to sensitive counterparts (p<0.05, FIG. 5A). Additionally, there was a 50.8% decrease in the level of caspase-3 activity (from 352.7±5.2 to 168.0±3.3 μM pNA) in cisplatin resistant A2780 EOC cells as compared to sensitive counterparts (p<0.001, FIG. 5).

3.6 Silencing MPO expression increased apoptosis in sensitive and chemoresistant EOC cells: Silencing MPO gene expression, utilizing specific siRNA for MPO resulted in an increase in caspase-3 activity as evident by decreased caspase-3 heavy and light chain S-nitrosylation of caspase-3 MDAH-2774 and SKOV-3 cells (FIG. 5B). There was a 34.6%±0.5, 34.3%±0.1, and 46.9%±1.1 increase in caspase-3 activity in sensitive, docetaxel, and cisplatin resistant MDAH-2774 EOC cells as compared to their scrambled siRNA controls (p<0.05, FIG. 5B). Similarly, there was a 54.5%±0.4, 34.6%±2.2, and 31.2%±2.2 increase in caspase-3 activity in sensitive, docetaxel, and cisplatin resistant SKOV-3 EOC cells as compared to their scrambled siRNA controls (p<0.05, FIG. 5B).

### D. Discussion

In this study, the inventors report the discovery of a novel integrin heterodimer αV/β1 that is specifically expressed in sensitive and chemoresistant EOC cells, with significantly higher expression in chemoresistant cells. To date, there is no report of presence of this specific integrin αV/β1 heterodimer in ovarian cancer cells.

The results presented here demonstrate that chemoresistant EOC cells manifest significantly lower levels of apoptosis as compared to their sensitive counterparts, highlighting a potential mechanism of decreased apoptosis mediated by integrin αV and integrin β1 (FIG. 5). Here, the inventors targeted integrin αV or B1 with their specific monoclonal antibody and observed a comparable cytotoxic effect in both sensitive and chemoresistant EOC cell lines (FIG. 4). One would expect enhanced cytotoxicity in chemoresistant EOC cells as compared to their sensitive counterparts, which should correlate with the observed enhanced expression in chemoresistant EOC cells. A possible explanation for this finding may be attributed to the fact that sensitive and chemoresistant EOC cells indeed have comparable cell surface expression of integrin αV and β1, despite enhanced integrin αV and β1 total protein levels in chemoresistant EOC cells (FIGS. 1 and 2). These results have also shown that macrophages only express integrin β1 and lack integrin αV (FIG. 2). Interestingly, treatment of macrophages with integrin αV or B1 antibody was not cytotoxic which may be attributed to the lack of integrin αV expression in macrophages (FIG. 4A). This suggests that both integrins are required to elucidate the cytotoxic effect observed in EOC cells. Additionally, the lack of an additive cytotoxic effect when treated with the combined antibody in both sensitive and chemoresistant EOC cells suggests that αV and β1 integrins exist as a heterodimer, where blocking one subunit is sufficient to initiate the cytotoxic effect.

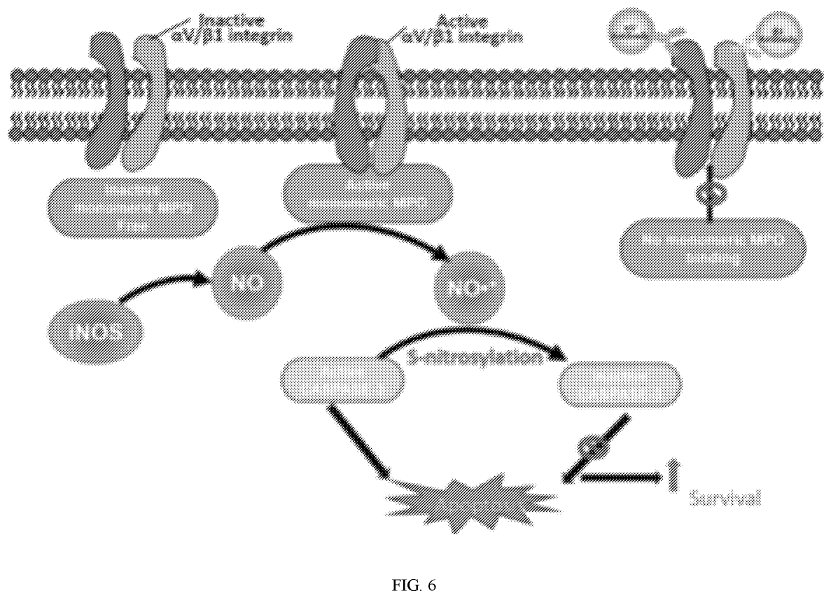

The inventors hypothesize that monoclonal antibodies against αV or β1 induce conformational changes that prevent monomeric MPO binding to αV/β1 integrin, thus preventing the activation of monomeric MPO and inducing cytotoxicity in EOC cells. They further hypothesize that binding of monomeric MPO to αV/β1 activates monomeric MPO in EOC cells. Activated monomeric MPO utilizes nitric oxide (NO), produced by iNOS, as a one-electron substrate to generate nitrosonium cation (NO+), a labile nitrosylating species that increases S-nitrosylation of caspase-3 and inhibits apoptosis and thus increases survival (FIG. 6).

These findings utilizing EOC cell lines are significant, as they further delineate a mechanism of ovarian cancer cell survival and thus, reveal a new target for the treatment of ovarian cancer. Moreover, the data showed synergistic effect of targeting αV/β1 receptor with chemotherapy. This chemosensitizing effects could allow for the use of lower doses of chemotherapy, highlighting an immense potential therapeutic value of this target.

All of the methods disclosed and claimed herein can be made and executed without undue experimentation in light of the present disclosure. While the compositions and methods of this invention have been described in terms of preferred embodiments and aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
[1] B. Zand, R.L. Coleman, A. K. Sood, Targeting angiogenesis in gynecologic cancers, Hematol. Oncol. Clin. North Am. 26 (2012) 543-63, viii. https://doi.org/10.1016/j.hoc.2012.01.009
[2] J.S. Berek, R.C. Bast Jr, Epithelial Ovarian Cancer, BC Deck., 2003.
[3] D. Jelovac, D. K. Armstrong, Recent progress in the diagnosis and treatment of ovarian cancer, CA: A Cancer J. for Clin. 61 (2011) 183-203. https://doi.org/10.3322/caac.20113.
[4] K. Matsuo, M.L. Eno, D.D. Im, N.B. Rosenshein, A. K. Sood, Clinical relevance of extent of extreme drug resistance in epithelial ovarian carcinoma, Gynecol. Oncol. 116 (2010) 61-65. https://doi.org/10.1016/j.ygyno.2009.09.018
[5] F. Aoudjit, K. Vuori, Integrin signaling in cancer cell survival and chemoresistance, Chemother. Res. Pract. 2012 (2012) 283181. https://doi.org/10.1155/2012/283181
[6] D. Naci, K. Vuori, F. Aoudjit, Alpha2betal integrin in cancer development and chemoresistance, Semin. Cancer Biol. 35 (2015) 145-153. https://doi.org/10.1016/j.semcancer.2015.08.004
[7] G. M. Saed, N. M. Fletcher, M.P. Diamond, R.T. Morris, N. Gomez-Lopez, I. Memaj, Novel expression of CD11b in epithelial ovarian cancer: Potential therapeutic target, Gynecol. Oncol. 148 (2018) 567-575. https://doi.org/10.1016/j.ygyno.2017.12.018
[8] J.S. Desgrosellier, D. A. Cheresh, Integrins in cancer: biological implications and therapeutic opportunities, Nat. Rev. Cancer. 10 (2010) 9-22. https://doi.org/10.1038/nrc2748
[9] Z. Liu, F. Wang, X. Chen, Integrin avB3-targeted cancer therapy, Drug Dev. Res. 69 (2008) 329-339. https://doi.org/10.1002/ddr.20265
[10] A.-F. Blandin, G. Renner, M. Lehmann, I. Lelong-Rebel, S. Martin, M. Dontenwill, B1 Integrins as Therapeutic Targets to Disrupt Hallmarks of Cancer, Front. Pharmacol. 6 (2015) 279. https://doi.org/10.3389/fphar.2015.00279
[11] M. Kobayashi, K. Sawada, T. Kimura, Potential of Integrin Inhibitors for Treating Ovarian Cancer: A Lit. Rev., Cancers. 9 (2017). https://doi.org/10.3390/cancers9070083.
[12] E. Lengyel, Ovarian cancer development and metastasis, Am. J. Pathol. 177 (2010) 1053-1064. https://doi.org/10.2353/ajpath.2010.100105
[13] S. Maubant, S. Cruct-Hennequart, S. Dutoit, Y. Denoux, H. Crouet, M. Henry-Amar, P. Gauduchon, Expression of a V-associated integrin β subunits in epithelial ovarian cancer and its relation to prognosis in patients treated with platinum-based regimens, J. Mol. Histol. 36 (2005) 119-129. https://doi.org/10.1007/s10735-004-4273-0
[14] S. A. Cannistra, C. Ottensmeier, J. Niloff, B. Orta, J. DiCarlo, Expression and function of beta 1 and alpha v beta 3 integrins in ovarian cancer, Gynecol. Oncol. 58 (1995) 216-225. https://doi.org/10.1006/gyno.1995.1214
[15] U. H. Weidle, F. Birzele, G. Kollmorgen, R. Rueger, Mechanisms and Targets Involved in Dissemination of Ovarian Cancer, Cancer Genom. [9] Protcom. 13 (2016) 407-423. https://doi.org/10.21873/cgp.20004
[16] N. Ahmed, F. Pansino, R. Clyde, P. Murthi, M. A. Quinn, G.E. Rice, M. V. Agrez, S. Mok. M.S. Baker, Overexpression of alpha (v) beta6 integrin in scrous epithelial ovarian cancer regulates extracellular matrix degradation via the plasminogen activation cascade, Carcinogen. 23 (2002) 237-244. https://doi.org/10.1093/carcin/23.2.237
[17] G. van der Horst, L. Bos, M. van der Mark, H. Cheung, B. Heckmann, P. Clément-Lacroix, G. Lorenzon, R.C. M. Pelger, R.F. M. Bevers, G. van der Pluijm, Targeting of alpha-v integrins reduces malignancy of bladder carcinoma, PLOS One. 9 (2014) c108464. https://doi.org/10.1371/journal.pone.0108464
[18] Y.-C. Lec, J.-K. Jin, C.-J. Cheng. C.-F. Huang, J.H. Song, M. Huang, W.S. Brown, S. Zhang, L.-Y. Yu-Lec, E.T. Ych, B.W. McIntyre, C.J. Logothetis, G.E. Gallick, S.-H. Lin, Targeting constitutively activated β1 integrins inhibits prostate cancer metastasis, Mol. Cancer Res. 11 (2013) 405-417. https://doi.org/10.1158/1541-7786. MCR-12-0551
[19] J. M. Rac, C. J. Creighton, J. M. Meck, B. R. Haddad, M. D. Johnson, MDA-MB-435 cells are derived from M14 Melanoma cells—a loss for breast cancer, but a boon for melanoma research, Breast Cancer Res. and Treat. 104 (2007) 13-19. https://doi.org/10.1007/s10549-006-9392-8.
[20] M. Bednarczyk, H. Stege, S. Grabbe, M. Bros, B2 Integrins-Multi-Functional Leukocyte Receptors in Health and Disease, Int. J. of Mol. Sci. 21 (2020) 1402. https://doi.org/10.3390/ijms21041402.
[21] D. Lau, H. Mollnau, J. P. Eiscrich, B. A. Freeman, A. Daiber, U. M. Gehling, J. Brümmer, V. Rudolph, T. Münzel, T. Heitzer, T. Meinertz, S. Baldus, Myeloperoxidase mediates neutrophil activation by association with CD11b/CD18 integrins, Proc. Natl. Acad. Sci. U.S.A 102 (2005) 431-436. https://doi.org/10.1073/pnas.0405193102
[22] G. M. Saed, R. Ali-Fehmi, Z. L. Jiang, N. M. Fletcher, M.P. Diamond, H. M. Abu-Soud, A. R. Munkarah, Myeloperoxidase serves as a redox switch that regulates apoptosis in epithelial ovarian cancer, Gynecol. Oncol. 116 (2010) 276-281. https://doi.org/10.1016/j.ygyno.2009.11.004
[23] N. M. Fletcher, Z. Jiang, R. Ali-Fehmi, N. K. Levin, J. Belotte, M. A. Tainsky, M. P. Diamond, H. M. Abu-Soud, G. M. Saed, Myeloperoxidase and free iron levels: potential biomarkers for early detection and prognosis of ovarian cancer, Cancer Biomark. 10 (2011) 267-275. https://doi.org/10.3233/CBM-2012-0255
[24] D. C. Castillo-Tong, D. Pils, G. Heinze, I. Braicu, J. Sehouli, A. Reinthaller, E. Schuster, A. Wolf, R. Watrowski, R. A. Maki, R. Zeillinger, W. F. Reynolds, Association of myeloperoxidase with ovarian cancer, Tumor Biol. 35 (2014) 141-148. https://doi.org/10.1007/s13277-013-1017-3
[25] H. M. Abu-Soud, S. L. Hazen, Nitric oxide modulates the catalytic activity of myeloperoxidase, J. Biol. Chem. 275 (2000) 5425-5430. https://doi.org/10.1074/jbc.275.8.5425
[26] J. Wang. J.-Y. Zhou, L. Zhang, G. S. Wu, Involvement of MKP-1 and Bcl-2 in acquired cisplatin resistance in ovarian cancer cells, Cell Cycle. 8 (2009) 3191-3198. https://doi.org/10.4161/cc.8.19.9751.
[27] A. Bellucci, C. Fiorentini, M. Zaltieri, C. Missale, P. Spano, The “In Situ” Proximity Ligation Assay to Probe Protein-Protein Interactions in Intact Tissues, Methods in Mol. Biol. (2014) 397-405. https://doi.org/10.1007/978-1-4939-0944-5_27.
[28] J. I. Greenberg, D. J. Shields, S. G. Barillas, L. M. Acevedo, E. Murphy, J. Huang, L. Scheppke, C. Stockmann, R. S. Johnson, N. Angle, D. A. Cheresh, A role for VEGF as a negative regulator of pericyte function and vessel maturation, Nat. 456 (2008) 809-813. https://doi.org/10.1038/nature07424.
[29] J. Fanning, W. C. Biddle, M. Goldrosen, K. Crickard, U. Crickard, M. S. Piver, K. A. Foon, Comparison of cisplatin and carboplatin cytotoxicity in human ovarian cancer cell lines using the MTT assay, Gynecol. Oncol. 39 (1990) 119-122. https://doi.org/10.1016/0090-8258 (90) 90416-i
[30] N. M. Fletcher, J. Belotte, M. G. Saed, I. Memaj, M. P. Diamond, R. T. Morris, G. M. Saed, Specific point mutations in key redox enzymes are associated with chemoresistance in epithelial ovarian cancer, Free Radic. Biol. Med. 102 (2017) 122-132. https://doi.org/10.1016/j.freeradbiomed.2016.11.028
[31] G. A. Howe, C. L. Addison, B1 integrin: an emerging player in the modulation of tumorigenesis and response to therapy, Cell Adh. Migr. 6 (2012) 71-77. https://doi.org/10.4161/cam.20077
[32] Ö. D. Işeri, M. D. Kars, F. Arpaci, U. Gündüz, Gene expression analysis of drug-resistant MCF-7 cells: implications for relation to extracellular matrix proteins, Cancer Chemother. and Pharmacol. 65 (2010) 447-455. https://doi.org/10.1007/s00280-009-1048-z.

What is claimed:

1. A method for treating endometrial or ovarian hyperproliferative disorders in a subject, the method comprising administration of an inhibitor of αV/β1 integrin, an MPO inhibitor, and/or the combination of an αV/β1 integrin and an MPO inhibitor to the subject.

2. The method of claim 1, wherein the hyperproliferative disorder comprises ovarian cancer or epithelial ovarian cancer.

3. The method of claim 1, wherein the hyperproliferative disorder comprises endometriosis.

4. The method of any one of claims 1-3, wherein the inhibitor comprises an anti-αV/β1 integrin, anti-αV integrin, anti-β1 integrin antibody, an anti-MPO antibody or combinations thereof.

5. The method of any one of claims 1-4, wherein the inhibitor inhibits the interaction of αV/β1 integrin and monomeric MPO (mMPO).

6. The method of any one of claims 1-5, wherein the inhibitor comprises an oligonucleotide inhibitor.

7. The method of claim 6, wherein the oligonucleotide inhibitor comprises an isolated oligonucleotide that hybridizes with a nucleic acid molecule encoding the MPO, αV integrin, and/or β1 integrin gene.

8. The method of claim 6 or 7, wherein the oligonucleotide inhibitor is an siRNA, a double stranded RNA, a short hairpin RNA, or an antisense oligonucleotide.

9. The method of any one of claims 1-8, wherein the inhibitor comprises one or more of GLPG0187, Bexotegrast (PLN-74809), PLN-1474, Abituzumab, Intetumumab, Volociximab, or Cilengitide.

10. The method of any one of claims 1-9, wherein the subject has ovarian or endometrial cells that are positive for expression of monomeric MPO and/or αV/β1 integrin.

11. The method of any one of claims 1-10, wherein the subject has been determined to have mMPO and/or αV/β1 integrin positive cells.

12. The method of any one of claims 1-11, wherein the subject is female.

13. The method of any one of claims 1-12, wherein the subject is on hormone therapy.

14. The method of claim 13, wherein the hormone therapy comprises contraception or hormone replacement therapy.

15. The method of any one of claims 12-14, wherein the female is an adolescent, perimenopausal, or menopausal female.

16. The method of any one of claims 1-15, wherein the subject has received one or more prior therapies to treat the hyperproliferative disorder.

17. The method of claim 16, wherein the subject has been determined to be a non-responder or resistant to the one or more prior therapies.

18. The method of any one of claims 16-17, wherein the one or more prior therapies comprise an immunotherapy.

19. The method of claim 18, wherein the immunotherapy comprises a checkpoint inhibitor.

20. The method of claim 19, wherein the immunotherapy comprises Pembrolizumab.

21. The method of any one of claims 16-20, wherein the one or more prior therapies comprises a targeted antibody.

22. The method of claim 21, wherein the targeted antibody comprises Bevacizumab.

23. The method of any one of claims 16-22, wherein the one or more prior therapies comprises a chemotherapy.

24. The method of claim 23, wherein the chemotherapy comprises a platinum-based chemotherapy, a taxane-based chemotherapy, or a combination thereof.

25. The method of claim 24, wherein the chemotherapy comprises carboplatin, cisplatin, paclitaxel, or a combination thereof.

26. The method of claim 25, wherein the prior therapy comprises cisplatin.

27. The method of any one of claims 24-26 wherein the prior therapy comprises docetaxel.

28. The method of any one of claims 1-27, wherein the method comprises administration of an additional agent.

29. The method of claim 28, wherein the additional agent comprises a platinum-based chemotherapy, a taxane-based chemotherapy, or a combination thereof.

30. The method of claim 28 or 29, wherein the additional agent comprises cisplatin.

31. The method of any one of claims 28-30, wherein the additional agent comprises docetaxel.

32. The method of any one of claims 29-31, wherein the subject is sensitive or has been determined to be sensitive to platinum-based chemotherapy and/or taxane based chemotherapy.

33. The method of claim 32, wherein the platinum-based chemotherapy comprises carboplatin and/or the taxane-based chemotherapy comprises paclitaxel.

34. The method of claim 32, wherein the platinum-based chemotherapy comprises cisplatin and/or the taxane-based chemotherapy comprises docetaxel.

35. The method of any one of claims 28-34, wherein the additional agent comprises a PARP inhibitor, a targeted antibody, or combinations thereof.

36. The method of claim 35, wherein the PARP inhibitor comprises olaparib or niraparib.

37. The method of claim 35, wherein the targeted antibody comprises bevacizumab.

38. The method of any of claims 2-37, wherein the ovarian cancer comprises stage I, II, III, or IV ovarian cancer.

39. The method of any one of claims 2-38, wherein the ovarian cancer comprises metastatic ovarian cancer.

40. A method for evaluating a subject comprising detecting αV/β1 integrin in a biological sample from the subject.

41. The method of claim 40, wherein the biological sample comprises serum, plasma, or tissue sample.

42. The method of claim 41, wherein the biological sample comprises a serum or plasma sample.

43. The method of any one of claims 40-42, wherein the method further comprises detecting iron levels in the biological sample from the subject.

44. The method of claim 43, wherein detecting iron levels in the biological sample from the subject comprises using one or more of a serum iron test, a free iron test, a total iron-binding capacity (TIBC) test, or a ferritin test.

45. The method of any one of claims 40-44, wherein the method comprises detecting the αV/β1 integrin complex.

46. The method of any one of claims 40-45, wherein detecting αV/β1 integrin comprises using one or more antibodies that specifically binds αV integrin, β1 integrin, and/or αV/β1 integrin complex.

47. The method of any one of claims 40-46, wherein detecting αV/β1 integrin comprises an ELISA assay.

48. The method of any one of claims 40-47 wherein the subject is one that has one or more symptoms of endometriosis and/or ovarian cancer.

49. The method of any one of claims 40-48, wherein the subject has been diagnosed with an endometrial or ovarian hyperproliferative disorder.

50. The method of any one of claims 40-49, wherein the subject has been treated for an endometrial or ovarian hyperproliferative disorder, will be treated for an endometrial or ovarian hyperproliferative disorder, or is currently undergoing treatment for an endometrial or ovarian hyperproliferative disorder.

51. The method of any one of claims 40-50, wherein the subject is female.

52. The method of any one of claims 40-51, wherein the subject is on hormone therapy.

53. The method of claim 52, wherein the hormone therapy comprises contraception or hormone replacement therapy.

54. The method of any one of claims 51-53, wherein the female is an adolescent, perimenopausal, or menopausal female.

55. The method of any one of claims 40-54, wherein the method further comprises quantitating the level of monomeric αV/β1 integrin in the biological sample.

56. The method of claim 55, wherein the level of αV/β1 integrin is normalized.

57. The method of claim 55 or 56, wherein the level of αV/β1 integrin is compared to a control.

58. The method of any one of claims 55-57, wherein the level of αV/β1 integrin is determined to be greater than the control.

59. The method of any one of claims 55-57, wherein the level of αV/β1 integrin is determined to be less than the control.

60. The method of any one of claims 40-59, wherein the control comprises the level of αV/β1 integrin that is representative of the level of αV/β1 integrin in a biological sample from a subject with endometriosis.

61. The method of any one of claims 40-59, wherein the control comprises the level of αV/β1 integrin that is representative of the level of αV/β1 integrin in a biological sample from a subject with ovarian cancer.

62. The method of any one of claims 40-59, wherein the control comprises the level of αV/β1 integrin that is representative of the level of αV/β1 integrin in a biological sample from a subject without an endometrial or ovarian hyperproliferative disorders.

63. The method of any one of claims 40-62, wherein the method further comprises diagnosing the subject.

64. The method of claim 63, wherein the subject is diagnosed with ovarian cancer based on the determined level of αV/β1 integrin.

65. The method of claim 64, wherein the subject is diagnosed with stage I, II, III, or IV ovarian cancer based on the determined level of αV/β1 integrin.

66. The method of claim 64 or 65, wherein the method further comprises treating the subject for ovarian cancer.

67. The method of claim 63, wherein the subject is diagnosed with endometriosis based on the determined level of αV/β1 integrin.

68. The method of claim 67, wherein the method further comprises treating the subject for endometriosis.

69. A method for making a complex comprising contacting a biological sample with an antibody that binds to αV/β1 integrin.

70. The method of claim 69, wherein the biological sample comprises serum, plasma, or tissue sample.

71. The method of claim 70, wherein the biological sample comprises a serum or plasma sample.

72. The method of any one of claims 69-71 wherein the biological sample is from a subject that has one or more symptoms of endometriosis and/or ovarian cancer.

73. The method of any one of claims 69-72, wherein the biological sample is from a subject that has been diagnosed with an endometrial or ovarian hyperproliferative disorder.

74. The method of any one of claims 69-73, wherein the biological sample is from a subject that has been treated for an endometrial or ovarian hyperproliferative disorder, will be treated for an endometrial or ovarian hyperproliferative disorder, or is currently undergoing treatment for an endometrial or ovarian hyperproliferative disorder.

75. The method of any one of claims 69-74, wherein the biological sample is from a female subject.

76. The method of any one of claims 69-75, wherein the biological sample is from a subject on hormone therapy.

77. The method of claim 76, wherein the hormone therapy comprises contraception or hormone replacement therapy.

78. The method of any one of claims 75-77, wherein the female is an adolescent, perimenopausal, or menopausal female.

79. The method of any one of claims 69-78, wherein the method further comprises quantitating the level of αV/β1 integrin in the biological sample.

80. The method of claim 79, wherein the level of αV/β1 integrin is normalized.

81. The method of claim 79 or 80, wherein the level of αV/β1 integrin is compared to a control.

82. The method of any one of claims 79-81, wherein the level of αV/β1 integrin is determined to be greater than the control.

83. The method of any one of claims 79-81, wherein the level of αV/β1 integrin is determined to be less than the control.

84. The method of claim 82 or 83, wherein the control comprises the level of αV/β1 integrin that is representative of the level of αV/β1 integrin in a biological sample from a subject with endometriosis.

85. The method of claim 82 or 83, wherein the control comprises the level of αV/β1 integrin that is representative of the level of αV/β1 integrin in a biological sample from a subject with ovarian cancer.

86. The method of claim 82 or 83, wherein the control comprises the level of αV/β1 integrin that is representative of the level of αV/β1 integrin in a biological sample from a subject without an endometrial or ovarian hyperproliferative disorders.

87. A method for treating a subject with an endometrial or ovarian hyperproliferative disorder, the method comprising administering an treatment for the endometrial or ovarian hyperproliferative disorder to a subject that has had the level of αV/β1 integrin evaluated in a biological sample from the subject.

88. The method of claim 87, wherein the biological sample comprises serum, plasma, or tissue sample.

89. The method of claim 87 or 88, wherein the subject is female.

90. The method of any one of claims 87-89, wherein the subject is on hormone therapy.

91. The method of claim 90, wherein the hormone therapy comprises contraception or hormone replacement therapy.

92. The method of any one of claims 89-91, wherein the female is an adolescent, perimenopausal, or menopausal female.

93. The method of any one of claims 87-92, wherein the level of αV/β1 integrin in the biological sample from the subject has been quantitated.

94. The method of claim 93, wherein the level of αV/β1 integrin is normalized.

95. The method of any one of claims 87-94, wherein the subject has or has been determined to have a level of αV/β1 integrin in the biological sample that is greater than the level of a αV/β1 integrin in a control sample.

96. The method of any one of claims 87-94, wherein the subject has or has been determined to have a level of αV/β1 integrin in the biological sample that is less than the level of a αV/β1 integrin in a control sample.

97. The method of any one of claims 87-94, wherein the subject has or has been determined to have a level of αV/β1 integrin in the biological sample that is not significantly different than the level of a αV/β1 integrin in a control sample.

98. The method of any one of claims 87-97, wherein the control comprises the level of αV/β1 integrin that is representative of the level of αV/β1 integrin in a subject with endometriosis.

99. The method of any one of claims 87-97, wherein the control comprises the level of αV/β1 integrin that is representative of the level of αV/β1 integrin in a subject with ovarian cancer.

100. The method of any one of claims 87-97, wherein the control comprises the level of αV/β1 integrin that is representative of the level of αV/β1 integrin in a subject without an endometrial or ovarian hyperproliferative disorders.

101. The method of any one of claims 87-100, wherein the subject has been evaluated for iron levels.

102. The method of claim 101, wherein the subject has been evaluated for iron levels by having one or more of a serum iron test, a free iron test, a total iron-binding capacity (TIBC) test, or a ferritin test in a biological sample from the subject.

103. The method of claim 101 or 102, wherein the subject has been determined to have abnormal iron levels.

104. The method of claim 103, wherein the subject has been determined to have a lower than normal TIBC or higher than normal ferritin.

105. The method of claim 103, wherein the amount of free iron was determined to be higher than normal in the biological sample from the subject.

106. The method of any one of claims 87-105, wherein the hyperproliferative disorder comprises endometriosis.

107. The method of claim 106, wherein the treatment comprises hormone therapy, or surgery.

108. The method of any one of claims 87-104, wherein the hyperproliferative disorder comprises ovarian cancer.

109. The method of claim 108, wherein the cancer comprises with stage I, II, III, or IV ovarian cancer.

110. The method of claim 108 or 109, wherein the treatment comprises surgery, radiation, chemotherapy, hormone therapy, or targeted therapy.

111. A method of diagnosing or prognosing a subject with an endometrial or ovarian hyperproliferative disorder comprising
a) evaluating αV/β1 integrin in a biological sample from the subject;
b) comparing the measured level to control level or control samples; and
c) diagnosing or prognosing the subject with an endometrial or ovarian hyperproliferative disorder based on the measured level of αV/β1 integrin.

112. The method of claim 111, wherein the biological sample comprises serum, plasma, or tissue sample.

113. The method of claim 112, wherein the biological sample comprises a serum or plasma sample.

114. The method of any one of claims 111-113, wherein the method further comprises evaluating iron levels in a biological sample from the subject.

115. The method of claim 114, wherein detecting iron levels in the biological sample from the subject comprises using one or more of a serum iron test, a free iron test, a total iron-binding capacity (TIBC) test, or a ferritin test.

116. The method of any one of claims 111-115, wherein evaluating αV/β1 integrin comprises using one or more antibodies that specifically bind αV integrin, B1 integrin, or αV/β1 integrin complex.

117. The method of any one of claims 111-116, wherein evaluating αV/β1 integrin comprises an ELISA assay.

118. The method of any one of claims 111-117, wherein evaluating αV/β1 integrin comprises quantitating the level of αV/β1 integrin in the biological sample.

119. The method of claim 118, wherein the subject is diagnosed as not having an endometrial or ovarian hyperproliferative disorder when αV/β1 integrin: i) is not detected in the biological sample from the subject; ii) is not significantly different than a control, wherein the control comprises the level of αV/β1 integrin that is representative of the level of αV/β1 integrin in a biological sample from a subject without an endometrial or ovarian hyperproliferative disorders; or iii) is less than a control, wherein the control comprises the level of αV/β1 integrin that is representative of the level of αV/β1 integrin in a biological sample from a subject with an endometrial or ovarian hyperproliferative disorder.

120. The method of claim 118, wherein the subject is diagnosed as not having an endometrial or ovarian hyperproliferative disorder when
(A) αV/β1 integrin:
i) is not detected in the biological sample from the subject;
ii) is not significantly different than a control, wherein the control comprises the level of αV/β1 integrin that is representative of the level of αV/β1 integrin in a biological sample from a subject without an endometrial or ovarian hyperproliferative disorders; or
iii) is less than a control, wherein the control comprises the level of αV/β1 integrin that is representative of the level of αV/β1 integrin in a biological sample from a subject with an endometrial or ovarian hyperproliferative disorder; and
(B) when iron levels are normal.

121. The method of claim 118, wherein the subject is diagnosed as having endometriosis when αV/β1 integrin: i) is greater than a control, wherein the control comprises the level of αV/β1 integrin that is representative of the level of αV/β1 integrin in a biological sample from a subject without an endometrial or ovarian hyperproliferative disorders; ii) is not significantly different than the control; wherein the control comprises the level of αV/β1 integrin that is representative of the level of αV/β1 integrin in a biological sample from a subject with endometriosis; or iii) is less than a control, wherein the control comprises the level of αV/β1 integrin that is representative of the level of αV/β1 integrin in a biological sample from a subject with ovarian cancer.

122. The method of claim 118, wherein the subject is diagnosed as having endometriosis when
(A) αV/β1 integrin:
i) is greater than a control, wherein the control comprises the level of αV/β1 integrin that is representative of the level of αV/β1 integrin in a biological sample from a subject without an endometrial or ovarian hyperproliferative disorders;
ii) is not significantly different than the control; wherein the control comprises the level of αV/β1 integrin that is representative of the level of αV/β1 integrin in a biological sample from a subject with endometriosis; or
iii) is less than a control, wherein the control comprises the level of αV/β1 integrin that is representative of the level of αV/β1 integrin in a biological sample from a subject with ovarian cancer; and
(B) when iron levels are abnormal.

123. The method of claim 122, wherein the TIBC is lower than normal and/or ferritin is higher than normal in the biological sample from the subject.

124. The method of claim 122 or 123, wherein the amount of free iron is higher than normal in the biological sample from the subject.

125. The method of claim 118, wherein the subject is diagnosed as having ovarian cancer when αV/β1 integrin: i) is greater than a control, wherein the control comprises the level of αV/β1 integrin that is representative of the level of αV/β1 integrin in a biological sample from a subject without an endometrial or ovarian hyperproliferative disorders or from a subject with endometriosis; ii) is not significantly different than a control, wherein the control comprises the level of αV/β1 integrin that is representative of the level of αV/β1 integrin in a biological sample from a subject with ovarian cancer.

126. The method of claim 118, wherein the subject is diagnosed as having ovarian cancer when
(A) αV/β1 integrin:
i) is greater than a control, wherein the control comprises the level of αV/β1 integrin that is representative of the level of αV/β1 integrin in a biological sample from a subject without an endometrial or ovarian hyperproliferative disorders or from a subject with endometriosis; or
ii) is not significantly different than a control, wherein the control comprises the level of αV/β1 integrin that is representative of the level of αV/β1 integrin in a biological sample from a subject with ovarian cancer; and
(B) when iron levels are abnormal.

127. The method of claim 126, wherein the TIBC is lower than normal and/or ferritin is higher than normal in the biological sample from the subject.

128. The method of claim 126 or 127, wherein the amount of free iron is higher than normal in the biological sample from the subject.

129. The method of any one of claims 125-128, wherein the subject is diagnosed with stage I, II, III, or IV ovarian cancer based on the measured level of αV/β1 integrin.

130. The method of claim 129, wherein evaluating αV/β1 integrin comprises qualitatively detecting the level of αV/β1 integrin in the biological sample.

131. The method of claim 130, wherein the subject is diagnosed as not having an endometrial or ovarian hyperproliferative disorder when αV/β1 integrin in not detected in the biological sample from the subject.

132. The method of claim 129, wherein the subject is diagnosed as having an endometrial or ovarian hyperproliferative disorder when αV/β1 integrin is detected in the biological sample from the subject.

133. The method of any one of claims 111-132, wherein the subject is one that has one or more symptoms of endometrial or ovarian hyperproliferative disorders.

134. The method of any one of claims 111-133, wherein the subject is female.

135. The method of any one of claims 111-134, wherein the subject is on hormone therapy.

136. The method of claim 135, wherein the hormone therapy comprises contraception or hormone replacement therapy.

137. The method of any one of claims 134-136, wherein the female is an adolescent, perimenopausal, or menopausal female.

138. The method of any one of claims 118-137, wherein the level of αV/β1 integrin is normalized.

139. The method of any one of claim 121-123 or 132-138, wherein the method further comprises treating the subject for endometriosis.

140. The method of claim 139, wherein the treatment comprises hormone therapy, or surgery.

141. The method of any one of claims 125-138, wherein the method further comprises treating the subject for ovarian cancer.

142. The method of claim 141, wherein the treatment comprises surgery, radiation, chemotherapy, hormone therapy, or targeted therapy.

143. A method for monitoring a subject being treated for an endometrial or ovarian hyperproliferative disorder with a therapeutic agent, the method comprising
a) evaluating αV/β1 integrin in a biological sample from the subject;
b) comparing the measured level to control level or control samples; and
c) determining the efficacy of the therapeutic agent based on the measured level of αV/β1 integrin.

144. The method of claim 143, wherein the biological sample comprises serum, plasma, or tissue sample.

145. The method of claim 144, wherein the biological sample comprises a serum or plasma sample.

146. The method of any one of claims 143-145, wherein the method further comprises evaluating iron levels in a biological sample from the subject.

147. The method of claim 146, wherein detecting iron levels in the biological sample from the subject comprises using one or more of a serum iron test, a free iron test, a total iron-binding capacity (TIBC) test, or a ferritin test.

148. The method of any one of claims 143-147, wherein evaluating αV/β1 integrin comprises using one or more antibodies that specifically bind αV integrin, β1 integrin, or the αV/β1 integrin complex.

149. The method of any one of claims 143-148, wherein evaluating αV/β1 integrin comprises an ELISA assay.

150. The method of any one of claims 143-149, wherein evaluating αV/β1 integrin comprises quantitating the level of αV/β1 integrin in the biological sample.

151. The method of claim 150, wherein the therapeutic agent is determined to be effective when αV/β1 integrin: i) is not detected in the biological sample from the subject; ii) is not significantly different than a control, wherein the control comprises the level of αV/β1 integrin that is representative of the level of αV/β1 integrin in a biological sample from a subject without an endometrial or ovarian hyperproliferative disorders; iii) is less than a control, wherein the control comprises the level of αV/β1 integrin that is representative of the level of αV/β1 integrin in a biological sample from a subject with an endometrial or ovarian hyperproliferative disorder; or iv) is decreased compared to the level of αV/β1 integrin before treatment of the subject with the therapeutic agent.

152. The method of claim 150, wherein the therapeutic agent is determined to be ineffective when αV/β1 integrin: i) detected in the biological sample from the subject; ii) is increased compared to a control, wherein the control comprises the level of αV/β1 integrin that is representative of the level of αV/β1 integrin in a biological sample from a subject without an endometrial or ovarian hyperproliferative disorders; iii) is not significantly different or more than a control, wherein the control comprises the level of αV/β1 integrin that is representative of the level of αV/β1 integrin in a biological sample from a subject with an endometrial or ovarian hyperproliferative disorder; or iv) is not significantly different or increased compared to the level of αV/β1 integrin before treatment of the subject with the therapeutic agent.

153. The method of any one of claims 143-152, wherein evaluating αV/β1 integrin comprises qualitatively detecting the level of αV/β1 integrin in the biological sample.

154. The method of any one of claims 143-153, wherein the subject is one that has one or more symptoms of endometrial or ovarian hyperproliferative disorders.

155. The method of any one of claims 143-154, wherein the subject has been diagnosed with an endometrial or ovarian hyperproliferative disorder.

156. The method of any one of claims 143-155, wherein the method further comprises evaluating the level of αV/β1 integrin in a biological sample from the subject obtained prior to treatment.

157. The method of any one of claims 143-156, wherein the method further comprises evaluating the level of αV/β1 integrin in a biological sample from the subject obtained after one or more treatments.

158. The method of any one of claims 143-157, wherein the subject is female.

159. The method of any one of claims 143-158, wherein the subject is on hormone therapy.

160. The method of claim 159, wherein the hormone therapy comprises contraception or hormone replacement therapy.

161. The method of any one of claims 158-160, wherein the female is an adolescent, perimenopausal, or menopausal female.

162. The method of any one of claims 150-161, wherein the level of αV/β1 integrin is normalized.

163. The method of any one of claims 143-162, wherein the treatment comprises hormone therapy, surgery, radiation, chemotherapy, or targeted therapy.

164. A kit comprising one or more anti-αV/β1 integrin, anti-αV integrin, and/or anti-β1 integrin antibodies or antigen binding fragments thereof.

165. The kit of claim 164, wherein the kit further comprises one or more negative or positive control samples.

166. The kit of claim 164 or 165, wherein the kit comprises an ELISA for αV/β1 integrin complex.

167. The kit of any one of claims 164-166, wherein the one or more antibodies or binding fragment is operatively linked to a solid support.

168. The kit of any one of claims 164-167, wherein the one or more antibodies or binding fragments are linked to a detectable label.
